SEQUENCE LISTING (9 sequences)

SEQ ID NO: 1 - DNA, 81 bp, Synthetic construct
  source 1..81: mol_type = other DNA
  misc_feature <1: note = 5' Phosphorylation
atgaagccaaggctggtgggtcgacagcagcttcaacattcgttagtcgaatcagtcctgtccgagaaaacgagacatgcc

SEQ ID NO: 2 - DNA, 45 bp, Synthetic construct
  source 1..45: mol_type = other DNA
  misc_feature <1: note = 5' Dibenzocyclooctyl:Triethylene glycol
  misc_feature 42^43: note = Phosphorothioate bond
  misc_feature 43^44: note = Phosphorothioate bond
  misc_feature 44^45: note = Phosphorothioate bond
aaaaaaactcctctggcaccgtgctgccttggcttcattgtggtc

SEQ ID NO: 3 - DNA, 133 bp, Synthetic construct
  source 1..133: mol_type = other DNA
aaaacgagacatgccgagcatccgccgcgcttcaattccttgttactgataggctgttgctaaagcattttgcagctcttctcggtttgaccacaatgaagccaaggcagcacggtgccagaggagttttttt

SEQ ID NO: 4 - DNA, 150 bp, Synthetic construct
  source 1..150: mol_type = other DNA
aaaacgagacatgccgagcatccgctgcgggtattacttagacctgttctggtgcctgagcttgggctggtggtccttaagccgggccgtgaatccatacagatagaccacaatgaagccaaggcagcacggtgccagaggagttttttt

SEQ ID NO: 5 - DNA, 21 bp, Synthetic construct
  source 1..21: mol_type = other DNA
  misc_feature <1: note = 5' terminal Cy3 fluorophore
caacgattgctcgagaatcat

SEQ ID NO: 6 - DNA, 24 bp, Synthetic construct
  source 1..24: mol_type = other DNA
  misc_feature <1: note = 5' terminal Cy5 fluorophore
tgcgggtattacttagacctgttc

SEQ ID NO: 7 - DNA, 104 bp, Synthetic construct
  source 1..104: mol_type = other DNA
  misc_feature <1: note = 5' phosphorylation
gaccacaatgaagccaaggctcgacagcagcttcaacattcgttagtcgaatcagtcctgtccgaggtattcttgcgagtctaataaaacgagacatgccgagc

SEQ ID NO: 8 - DNA, 190 bp, Synthetic construct
  source 1..190: mol_type = other DNA
  misc_feature <1: note = 5' phosphorylation
atccgctgcgggtattacttagacctgttctggtgcctgagcttgggctggtggtccttaagccgggccgtgaatccatacagatagaccacaatgaagccaaggctcgacagcagcttcaacattcgttagtcgaatcagtcctgtccgaggtattcttgcgagtctaataaaacgagacatgccgagc

SEQ ID NO: 9 - DNA, 40 bp, Synthetic construct
  source 1..40: mol_type = other DNA
aagtaatacccgcagcggatgctcggcatgtctcgtttta

(19) United States
(12) Patent Application Publication
(10) Pub. No.: US 2026/0234707 A1
(43) Pub. Date: Aug. 13, 2026

(54) CLONAL AMPLIFICATION

(71) Applicant: DNAe Diagnostics Limited, London (GB)

(72) Inventors: David Wooldridge, London (GB); Dominic Lovedale, London (GB)

(21) Appl. No.: 19/122,951

(22) PCT Filed: Oct. 20, 2023
(86) PCT No.: PCT/GB2023/052756

§ 371 Date: Apr. 21, 2025

(30) Foreign Application Priority Data
Oct. 21, 2022 (GB) 2215624.4

(51) Int. Cl. C12Q 1/6844; C12Q 1/6837

(52) U.S. Cl. CPC C12Q 1/6844; C12Q 1/6837

(57) ABSTRACT

The present invention provides methods for the preparation of clonal clusters of polynucleotides on surfaces and also provides surfaces comprising such clusters. The methods and tethered clonal clusters are particularly of use in nucleotide sequencing methodologies such as sequencing by synthesis.

The present invention relates, inter alia, to methods for clonal amplification of nucleotide sequences of interest, (SOIs), to methods of nucleotide sequencing incorporating these approaches and to surfaces comprising clonally amplified nucleotide sequences.

In solid phase sequencing workflows, individual nucleotide fragments of interest are typically captured by an oligonucleotide immobilized on a surface such as a slide, a bead or a feature in a microfluidics system. Sequencing reactions are then carried out on the tethered molecule. This allows for i.a. the use of very small quantities of material, simple control of the necessary fluids and the ability to carry out many similar reactions arranged in a small space. In recent approaches to nucleotide sequencing, a sequencing primer is annealed to the tethered sequence and as the primer is extended, the incorporation of each base in turn is detected (the incorporation signal), for example by fluorescence or detection of ions generated in the reaction. In order to increase the incorporation signal, the single tethered molecules may be amplified to generate clonal clusters of tethered amplicons, originating from a single polynucleotide. This “clonal amplification” (CA) provides many duplicated sequencing targets and so allows multiple incorporation events to occur at the same time within the cluster, thereby amplifying the signal many times over. A number of approaches to CA are known and have been developed and commercialized for use, for example, in sequencing workflows. These include bridge PCR (U.S. Pat. No. 9,593,328), emulsion PCR (US20100261230 A1) and kinetic exclusion amplification (U.S. Pat. No. 9,169,513). Clonal amplification by recombinase polymerase amplification (RPA) has also been reported and is discussed in co-pending application GB2110479.9 and further herein below.

Although CA methodologies typically provide an increased signal, the level of the signal and/or its level above background may still be challenging in some cases. Optimising particular CA methodologies can improve the density of tethered amplicons but density, and hence signal, may still be limited by factors, such as the density of capture oligos immobilised on the surface and stearic considerations reducing access of polymerases to locally crowded molecules during clonal amplification. Further, approaches to increasing the nucleotide or target density within tethered clusters may also affect cluster spread and may increase the incidence of contamination from neighbouring amplification events and reduce clonality, hamper read quality, increase background and adversely affect signal to noise ratio, particularly around the edges of the clusters. By increasing the density of clonal clusters it may also be possible to reduce the space each cluster occupies and therefore provide scope for increasing the number of clusters in a given area without reducing the signal generated by each cluster.

In some circumstances it may be desirable to provide clusters based on sequences that are known, or partially known, for example where polymorphisms within the sequence are to be determined by sequencing, or where the presence of particular nucleotide sequences within a sample will be determined. In some circumstances it may be desirable to provide clonal clusters of unknown sequences, for example where genomic fragments are to be sequenced for later assembly, or where other unknown nucleotide sequences are to be determined.

It would therefore be desirable to provide improved methods of clonal amplification. It would also be desirable to do so without adversely affecting clonality, cluster spread or signal to noise ratio. It would also be desirable to provide a clonal amplification method that could easily be adapted for use on known, partially known or unknown sequences. The present invention addresses one or more of the above issues.

In a first embodiment, the present invention provides a method for preparing a clonal cluster of a nucleotide sequence of interest comprising: (i) providing a single stranded polynucleotide comprising the nucleotide sequence of interest (SOI), wherein the polynucleotide is tethered to a surface through its 5′ end; (ii) carrying out a first clonal amplification of the single stranded polynucleotide to provide a first cluster of polynucleotides comprising a plurality of single stranded polynucleotides tethered to the surface through their 5′ ends and comprising the nucleotide sequence of interest; and (iii) extending tethered single stranded polynucleotides in the first cluster, by the addition of one or more further copies of the nucleotide sequence of interest to provide a cluster of tethered polynucleotides wherein polynucleotides of the cluster comprise a plurality of copies of the nucleotide sequence of interest.

A second embodiment provides a method for preparing a clonal cluster of a single stranded polynucleotide comprising a sequence of interest, the method comprising hybridizing a polynucleotide fragment comprising a complementary copy of the sequence of interest to a capture oligo the capture oligo being tethered to a surface through its 5′ end (ii) extending the 3′ end of the capture oligo using a polymerase to provide a single stranded polynucleotide comprising the sequence of interest, tethered to the surface through its 5′ end; (iii) carrying out a first clonal amplification of the tethered single stranded polynucleotide to provide a first cluster of polynucleotides comprising a plurality of single stranded polynucleotides tethered to the surface through their 5′ ends and comprising the nucleotide sequence of interest; and (iii) extending the 3′ end of tethered single stranded polynucleotides in the first cluster, by the addition of one or more further copies of the sequence of interest to provide a cluster of tethered polynucleotides wherein polynucleotides of the cluster comprise a plurality of copies of the nucleotide sequence of interest.

In one aspect the polynucleotide is hybridized to the capture oligo through a 5′ terminal portion. The polynucleotide may be provided with a 3′ adaptor and optionally a 5′ adaptor, and at least a portion of the 3′ adaptor is hybridized to the capture oligo.

In a third embodiment is provided a method for determining a nucleotide sequence of a sequence of interest comprising: (i) tethering the sequence of interest to a surface through its 5′ end; (ii) carrying out a first clonal amplification step to clonally amplify the SOI to provide a first clonal cluster of oligonucleotides each comprising a copy of the SOI. (iii) extending the single stranded polynucleotides in the first cluster, by the addition of one or more further copies of the SOI to provide a cluster of tethered polynucleotides wherein polynucleotides of the cluster comprise a plurality of copies of the nucleotide sequence of interest; and (iv) at least partially sequencing the polynucleotides of the cluster to determine a nucleotide sequence of the SOI.

The tethered SOI may be a complementary copy of a polynucleotide obtained by an earlier workflow step.

As referred to herein, the “sequence of interest” or SOI may be any nucleotide sequence which the user seeks to clonally amplify. As referred to herein the nucleotide sequence of the SOI is that of the polynucleotide tethered through its 5′ end before initial clonal amplification to provide the first clonal cluster. This sequence may be a complementary copy of a polynucleotide generated in earlier stages of a workflow as a result of the tethering process as described further herein.

Typically the SOI is a polynucleotide sequence about which the user seeks information. This may be sequence information, including the sequence or partial sequence of a polynucleotide or the presence or absence of a specific sequence or polymorphism for example. Alternatively it might be binding information such as the ability of a sequence to bind a particular binding partner, such as a primer or other polynucleotide, nucleotide binding protein or a drug for example.

The SOI may be any length required dependent on the parameters of the workflow and the information sought. Typically the SOI is from 1 base to 100,000 bases long. For example it may be 1 to 10,000 bases, preferably 5 to 1000 bases but is typically of the order of 10 to 500 bases or 50 to 250 bases.

In some cases the SOI may be a polynucleotide of a known sequence or it may be a polynucleotide of unknown sequence or of partially unknown sequence. The SOI may be a DNA or an RNA. For example it may be (without limitation) a fragment of a genomic DNA to be sequenced or it may be a polynucleotide covering a polymorphic region, a cDNA derived from an RNA sequence (such as an mRNA, rRNA or a tRNA) by reverse transcription, or a polynucleotide fragment whose sequence is diagnostic or indicative of a particular disease, condition or organism (such as an infectious agent). The skilled person will be aware that the sequence information obtained for a forward strand may be obtained from the sequence of the complementary (reverse) strand.

In some embodiments, a polynucleotide or an SOI is provided with adaptors. An adaptor may be a single stranded or double stranded nucleotide sequence added to the 3′ or 5′ terminus of a sequence of interest, but preferably to both ends. Adaptors typically are of known sequence and are designed to comprise separate or overlapping functional sequences, configured for certain downstream purposes.

Such sequences may hybridize to primers (eg sequencing primer or clonal amplification primers) to capture oligos or to complementary sequences in molecular inversion probes for example, or they may be configured to facilitate coupling to beads or to other surfaces. Further functional sequences include identification tags, such as nucleic acid bar codes. In separate functional sequences, no nucleotide of the sequence forms part of another functional sequence. In overlapping functional sequences nucleotides of one sequence may also be part of a second functional sequences, for example the n 5′ nucleotides of a first functional sequence may form the n 3′ nucleotides of a second functional sequence. Universal adaptors are adaptors borne by all polynucleotides in a population.

Adaptors may be provided as part of an upstream workflow (i.e. an earlier step) or provided separately for the purposes of clonal amplification. Workflows might include (in non-limiting examples) one or more steps such as isolation and fragmentation of a larger polynucleotide, such as genomic DNA, amplification of fragments, such as by PCR or by an isothermal amplification methodology, addition of adaptors to 5′ and/or 3′ ends of the fragment and so on. In some embodiments adaptors may be ligated to the 5′ and/or 3′ ends of a double stranded fragment, followed by denaturing to provide single stranded polynucleotides. Alternatively the adaptors may be incorporated via a tagmentation reaction in which a transposase, such as Tn5 both cleaves the DNA and appends short tags that may include adaptor sequence. In a further approach the adaptors may be incorporated during a step in which the fragment is amplified or duplicated using tailed primers.

Typically the surface is the surface of a substrate to which the polynucleotides are tethered. In non-limiting examples the substrate may be an ISFET, a glass or silica substrate, an insoluble particulate substrate Such as a micro or nano sphere), or a portion of a microfluidics device adapted to bring the tethered polynucleotides into contact with liquid reagents. The surface may be planar, such as the surface of a slide or semiconductor chip or it may be in the form of wells or other 3-D features. The surface/substrate may comprise a variety of materials including inorganic materials such as glass, silica or Ta₂O₅ or organic materials including a variety of polymers.

In one embodiment, the surface is the surface of a semiconductor chip comprising a field effect transistor (FET) array for sensing chemical and/or biological reactions, including sequencing reactions. Such devices are well known in the art (e.g., U.S. Pat. No. 7,686,929 B2; U.S. Pat. No. 8,685,228 B2; U.S. Pat. No. 8,986,525 B2; US 2010/0137143 A1). A preferred embodiment comprises a semiconductor chip comprising an ion sensitive field effect transistor (ISFET) array useful as a sensing device for various reactions, including nucleic acid sequencing reactions. In particularly preferred embodiments, the chip further comprises an array of wells positioned above the ISFET array and in fluid contact therewith. In these embodiments, the sequencing reaction typically occurs within the wells and the release of ions is detected by the ISFET sensors. In particularly preferred embodiments, the chip comprises a flow cell mounted on top of the chip (with or without wells) for the delivery of fluids to the chip/ISFET array and removal of fluids therefrom. The term surface also includes surfaces that have been modified in order to provide functional groups to which a polynucleotide may be coupled, including, without limitation, by directly functionalising the surface or by coating the surface with a polymer bearing suitable functional groups.

Polynucleotides may be tethered to a surface using a number of approaches, they may, for example, be chemically bonded to the surface, such as by covalent bonding, or they may be captured by a capture moiety that itself is bonded to the surface.

In some embodiments tethering may occur through an affinity tag, such as interaction between a protein or peptide and a cognate ligand thereof. Typically the ligand is attached to the polynucleotide and the protein or peptide is attached to the surface as the capture moiety. Such pairs include for example biotin and biotin binding partners such as avidin, NeutrAvidin™ (Thermo Fisher) or streptavidin, but many more are known, have been well studied and are commercially available. Biotin is particularly useful since biotinylation of nucleic acids is well known and the interaction between biotin and its binding partner is strong. Further, biotin tagged oligos, for example are available from a number of sources. In some cases surfaces may be coated to provide appropriate functional groups to which the polynucleotide or the capture moiety, such as a capture oligo, may be coupled.

Polynucleotides or oligos may be tethered through the 5′ or 3′ termini. tethering may occur through a functionalized 5′ or 3′ nucleotide. Typically in the present invention polynucleotides and oligos are tethered to the surface through their 5′ ends. The exact chemistry of attachment of the polynucleotide to the surface is dependent on the surface concerned and many chemistries are available commercially for this purpose, including a number of so-called “click chemistry” approaches (eg Click Chemistry, a Powerful Tool for Pharmaceutical Sciences (2008) Hein, et al.; Pharm Res 25(10): 2216-2230 and A Hitchhiker's Guide to Click-Chemistry with Nucleic Acids (2021) Fantoni et al., Chem Rev, 121: 7122-7154).

In some embodiments the tethered polynucleotide is provided by hybridization of the polynucleotide to a capture oligo and extending the capture oligo to provide a complementary copy of the polynucleotide tethered to the surface through its 5′ end. Hybridization may take place between complementary sequences of the 3′ portion of the capture oligo and the 3′ portion of the polynucleotide. The 3′ portion of the polynucleotide may comprise an adaptor comprising a sequence complementary to the 3′ “capture” region of the capture oligo.

Following hybridization, the 3′ end of the capture oligo acts as a primer and is extended over the captured polynucleotide using a polymerase to provide a double stranded polynucleotide. Denaturing the two strands then leaves a single stranded polynucleotide tethered to the surface through its 5′ end. This tethered polynucleotide comprises a complementary copy of the original, captured, polynucleotide, (including any 5′ and 3′ adaptors) which is now continuous with the capture oligo and hence is tethered to the surface through its 5′ end.

In some embodiments the surface is provided with a single population of capture oligos having an identical 3′ capture sequence. In other embodiments the surface may be provided with a mixed population of 2, 3, 4, or more capture oligos, each comprising a different 3′ capture sequence. For example the surface may be provided with a mixed population of two capture oligos, the first population comprising a 3′ sequence complementary to the 3′ adaptor (reverse primer), the second comprising a 3′ sequence complementary to the 3′ end of the extended complementary polynucleotide and acting as forward primer as described further herein.

The capture oligos may comprise nucleotide sequences or other features that provide additional functionality (other than the 3′ capture portion) such as spacer sequences or chemical spacers that distance the tethered portion from surface effects, for example, and chemically modified nucleotides or linkages at the 3′ end (such as phosphorothioate linkages) that protect the oligo against 3′ to 5′ exonuclease activity of polymerase enzymes.

In some embodiments, the single stranded polynucleotide may include, in a 5′ to 3′ direction, a 5′ flanking region, a nucleotide sequence comprising or consisting of the nucleotide sequence of interest and a 3′ flanking region. The 5′ and 3′ flanking regions are directly coupled to the 5′ and 3′ extremity of the sequence of interest, such that they are continuous with it. Typically the 5′ and 3′ flanking regions are adaptors.

In a preferred arrangement, the 5′ and 3′ flanking regions are of a known and predetermined sequence. Typically these are “universal adaptors”, that is to say that they are adaptors common to all oligo nucleotides applied to the surface. The 5′ and 3′ flanking regions or adaptors are typically of a different sequence. Each flanking region may comprise one or more sequences complementary to primers that are suitable for various functions. For example flanking regions or adaptors may comprise one or more regions complementary to clonal amplification primers and/or the 3′ flanking region may comprise a region complementary to a sequencing primer. As described further below, they may comprise distinct universal regions complementary to molecular inversion probe (MIP) 3′ and/or 5′ complementary regions. In some cases, these can be separate from other binding regions enabling the independent design of high specificity binding regions for MIP complementary regions.

In the case of the 3′ flanking region it is advantageous for the flanking region to be directly coupled to the 3′ end of the sequence of interest, such that the 5′ most nucleotide of the flanking region is bonded directly to the 3′ most nucleotide of the sequence of interest. A sequencing primer complementary to the adaptor sequence and whose 3′ end is co-terminal with the 5′ end of the adaptor, can initiate sequencing at the 3′ most end of the sequence of interest. Alternatively, a short known sequence of nucleotides in the flanking region between the 3′ end of the sequencing primer and the 5′ end of the sequence of interest may allow for an internal sequence-calling control. A short sequence of 1, 2, 3, 4, or 5 nucleotides, for example may be used. Other functional sequences within the flanking regions or adaptors include barcodes or other identification sequences.

In some embodiments the 3′ end of the 3′ flanking region is at the 3′ end of the tethered polynucleotide. In some embodiments the 3′ end of the 3′ flanking region is at the 3′ end of the tethered polynucleotide and co-terminal with it.

In some embodiments the 3′ end of the sequence of interest is at the 3′ end of the tethered polynucleotide. In some embodiments the 3′ end of the sequence of interest is at the 3′ end of the tethered polynucleotide and co-terminal with it. This might be the case for example if the sequence of interest has no 3′ flanking region.

In some embodiments a single species of polynucleotide may be spotted onto the surface in distinct regions. In other approaches, the polynucleotides may be applied to the surface by flooding the surface with a dilute solution of a population of polynucleotides which may comprise different sequences of interest. The dilution of the polynucleotides is calculated such that single polynucleotides are captured in a spatially separated manner, such that clonal amplification provides separated clusters. In some embodiments the surface comprises features such as wells intended (statistically) to capture a single polynucleotide.

Once tethered to the surface the polynucleotides can be clonally amplified to provide a first clonal cluster comprising a plurality of single stranded polynucleotides tethered to the surface through their 5′ ends and comprising the nucleotide sequence of interest. The first clonal amplification can be carried out using a number of methodologies well known in the art and selected according to need. A non-limiting selection of these is described below. Methods include (without limitation) bridge amplification (see U.S. Pat. No. 9,593,328), rolling circle amplification, kinetic exclusion amplification—exAMP (see U.S. Pat. No. 9,169,513), emulsion PCR (particularly suitable to use with microspheres see US20100261230 A1) and template walking (eg US2012/0156728). Clonal amplification by recombinase polymerase amplification (RPA) has also been reported and is discussed in co-pending application GB2110479.9. Any method of clonal amplification is suitable as long as it provides a clonal cluster comprising a plurality of preferably single stranded polynucleotides tethered to the surface through their 5′ ends. In some embodiments the method of clonal amplification chosen provides only sense copies of the SOI or antisense copies of the SOI but not both, in the first cluster. In some embodiments the method provides both sense and antisense copies of the SOI in the same first cluster. In some embodiments, polynucleotides of the first cluster comprise only a single copy of the SOI. In some embodiments, and preferably, the tethered polynucleotides comprise free 3′ ends. In one embodiment, the clonal amplification method provides a clonal cluster comprising a plurality of polynucleotides tethered to the surface through their 5′ ends, and having a free 3′ end, preferably comprising only one copy of the SOI.

In one approach to bridge amplification the fragment is provided with 5′ and 3′ adaptors. A mixed population of both forward and reverse primers are tethered to the surface. The single stranded template binds to the reverse primer on the surface through its 3′ adaptor and a polymerase extends the primer over the template to produce a tethered complementary copy of the template with its 3′ and 5′ adaptors. This duplex is then denatured (typically by exposing the duplex to a denaturing solution or heating to above the Tₘ for the duplex) to release the original template. The 5′ adaptor of the tethered complementary copy is then captured by the tethered forward primer to form a bridge bound to the surface. Extension of the forward primer using the tethered and bridged complementary strand provides a copy of the original single stranded fragment coupled to the surface through the second capture oligo. The double stranded bridge is then denatured leaving copies of both the forward and reverse strand tethered to the surface through their 5′ ends. Following washing, the 3′ end of these strands are then captured by tethered forward and reverse primers and a further cycles of extension and denaturation then take place, spreading the clonal cluster across the surface until clonal amplification is complete (see for example U.S. Ser. No. 10/370,652B2 and U.S. Pat. No. 7,972,820B2).

Clonal amplification by recombinase polymerase amplification (RPA) makes use of two primers, one of which (the reverse primer) is a capture oligo tethered on the surface, and the second (forward) is in solution. A polynucleotide fragment is provided with 5′ and 3′ adaptors. The 3′ adaptor hybridizes to the capture oligo and the 3′ end of the capture oligo is then extended over the polynucleotide template using a polymerase to provide a complementary copy of the polynucleotide tethered to the surface through its 5′ end, but free at its 3′ end. A reaction mixture including recombinase(s), a single stranded DNA binding protein and a strand displacing polymerase is then provided and concerted action of the recombinase(s) and single stranded DNA binding proteins separates the 3′ end of the duplex. This allows the forward (solution phase) primer access to the 3′ end of the tethered complementary strand and allows a strand displacing polymerase to extend the forward primer, eventually displacing the original fragment into solution. In some systems (eg T4) a recombination mediator protein may be provided to mediate access of the polymerase to the single stranded DNA. The released fragment is then free to hybridize to a further capture oligo to repeat the process. By increasing the viscosity of the reaction mixture, local diffusion of the released template can be reduced and so cluster spread can be reduced. This approach is discussed further in relation to FIG. 1.

In template walking (US2012/0156728) the capture oligo has a 3′ end of low Tₘ (in its reaction conditions), for example it can be a portion with a high proportion of A or T (or U). It can be for example a sequence of 20T. The SOI is provided with adaptors and the 3′ adaptor comprises a region complementary to the 3′ region of the capture oligo (eg 20 A or 30 A), the rest of the SOI has a Tm higher than the 3′ end of the adaptor. The polynucleotide with the 5′ and 3′ adaptors, is hybridized to the low Tₘ end of the capture oligo and the 3′ end of the capture oligo is extended using the polynucleotide as template to provide a complementary copy which is tethered to the surface through its 5′end. The duplex comprises a region of relatively high Tₘ and a region around the capture oligo with a low Tₘ The temperature is then raised to de hybridize the 3′ end of the original polynucleotide from the capture oligo without dissociating the whole duplex over the SOI. As the temperature is lowered, the low Tₘ region can re-hybridize to a neighbouring capture oligo, providing a template from which to once again extend the capture oligo and thus displace the original polynucleotide from the original, extended capture oligo.

Kinetic exclusion amplification (exAMP) was developed in response to issues of clonal contamination, particularly in well like features in which the workflow attempts to capture and amplify a single polynucleotide fragment per well on the surface and where capture and amplification proceed at the same time. If capture is rapid, but amplification is slow, then the probability is high that a second polynucleotide fragment of a different sequence will be captured close by, leading to clusters generated from the two different sequences over lapping and reducing clonality. Kinetic exclusion amplification attempts to maintain clonality by making clonal amplification outstrip the rate of capture of new polynucleotide species. This reduces the supply of local capture oligos and impedes access by a new polynucleotide species to the lawn of capture oligos.

One way to do this is provide the oligonucleotide fragments (in double stranded form) along with DNA polymerase, single stranded binding protein (ssBP) and recombinase. The concentration of the polynucleotides in solution is controlled such that the rate of capture of single stranded polynucleotide by capture oligo is much lower than the rate of clonal amplification and consequent exhaustion of the capture oligos close by.

The clonal amplification methods may provide clusters of only one strand (such as RPA) or of both forward and reverse strands (e.g. bridge amplification). It is preferred that the CA method chosen results in clusters of linear polynucleotides comprising the sequence of interest and having a free 3′ terminus. In one embodiment, the linear polynucleotides comprise a single copy of the SOI and have a free 3′ terminus. Clonal amplification by RPA is typically the preferred approach used in this invention.

Once the first clonal amplification is complete the surface comprises a cluster of polynucleotides arranged as a two dimensional feature (or “lawn”) on the surface, each polynucleotide comprising the sequence of interest. Typically, at this point each polynucleotide comprises only one copy of the SOI. However, by extending at least a portion of the tethered single stranded polynucleotides in the first cluster, by the addition of one or more further copies of the SOI, it's possible to provide a cluster of tethered polynucleotides which comprise a plurality of copies of the original nucleotide sequence of interest (or at least a portion of them do). In this way the number of copies of the sequence of interest in the cluster may be increased several fold without the need to tether further copies of the oligonucleotide to the surface, but the clonality is maintained. This approach overcomes the issue of increasing the number of SOIs per unit area in a manner that does not tend to spread the cluster. This tends to improve the signal by providing what is effectively a three-dimensional cluster, having multiple copies of the SOI and may also provide multiple copies of sequencing adaptors and other functional sequences useful in downstream processing of the SOI.

In one convenient embodiment the tethered, single stranded polynucleotides in the first cluster may be extended by rolling circle amplification (RCA) (of at least the SOI). RCA may be used to duplicate just the sequence of interest (or at least a portion thereof) or it may additionally duplicate all or part of the 5′ and/or 3′ adaptors as well. In this way, where present in the original polynucleotide, the method provides copies of sequencing adaptors positioned to initiate sequencing from each SOI, and likewise provides, where present in the original polynucleotide, copies of any other functional sequences useful in downstream processing of an SOI.

Extension by RCA can be achieved in a number of ways and can be applied whether the SOI has a known or partially known sequence or an unknown sequence, making this a useful approach for increasing the density of SOIs in a clonal cluster, which can be adapted to a variety of workflows.

In one embodiment the rolling circle amplification may be carried out by a process comprising: (a) providing a single stranded circular nucleotide probe comprising a nucleotide sequence which is complementary to the SOI, and optionally to 5′ and/or 3′ adaptors or parts thereof, in the first clonal cluster; and (b) using the circular nucleotide probe as a template, extending the 3′ end of the tethered single stranded polynucleotide, preferably using a strand displacing polymerase, thereby extending the 3′ end of the tethered single stranded polynucleotides (or at least a portion of them), by the addition of one or more further copies of the sequence of interest.

In some embodiments, the single stranded circular probe may be provided as a preformed circular probe, comprising a complementary copy of the sequence of interest. The complementary copy of the SOI is then hybridized to the tethered SOI, (and optionally to any 5′ or 3′ adaptors or portions thereof) and the 3′ end of the tethered oligonucleotide is then extended by repeated duplication of the circular probe in complement using a strand displacing polymerase. This approach is particularly useful where the SOI is of known sequence. Usefully, the circular probe may be prepared in solution, for example by ligating the free ends of a linear probe comprising a complementary copy of the SOI.

In a further modification of this approach the single stranded circular nucleotide probe comprises a nucleotide sequence which is complementary to the SOI, and flanking the SOI, complementary copies of 5′ and or 3′ adaptors whose sequence is not present in the tethered polynucleotide. RCA of the tethered polynucleotide will then incorporate functional adaptors, into the extending strand.

In either case, the circular probe may additionally comprise an optional linker region extending from the 3′ end of the sequence complementary to the SOI and optionally a 3′ adaptor, to the 5′ end of the SOI and optionally any 5′ adaptor. Such single stranded circular probes may be constructed in a number of ways, well known to those skilled in the art, and discussed elsewhere herein.

In some embodiments, the circular probe is not provided as a preformed circular probe, but is provided as a linear probe from which the circular probe may be synthesized in situ i.e. on the surface rather than as a preformed probe. In practice, this means that at least a portion of the linear probe is hybridized to the polynucleotide whilst the circular probe is formed and preferably the process of circularization occurs whilst the probe is hybridized to the polynucleotide.

In one approach in situ formation of the circular probe may be achieved by:
(a) providing a linear single stranded nucleotide probe comprising in a 5′ to 3′ direction, a 5′ complementary region (5′ CR), an optional linker region and a 3′ complementary region (3′ CR); wherein the 5′ CR and the 3′ CR are configured to hybridize to separate sequences (hybridization sites) on the tethered, single stranded polynucleotides;
(b) hybridizing the 5′ CR of the probe and to the 3′ CR of the probe to the tethered single stranded polynucleotide; wherein the 5′ CR hybridizes to a sequence that is up stream of (i.e. closer to the 5′ end of the tethered polynucleotide than) the sequence to which the 3′ CR hybridizes;
(c) optionally extending the 3′ end of the linear probe using the sequence of the single stranded polynucleotide as a template; and
(d) cyclizing the probe by ligating the optionally extended 3′ end of the probe to the 5′ end of the probe. Such linear probes may be referred to as “molecular inversion probes”

The sequence of the 5′ CR of the probe and of the 3′ CR of the probe are selected such that, following optional extension and ligation, a single stranded circular probe is provided comprising a complementary copy of the sequence of interest. The sequence of the 5′ and 3′ CRs can be selected in a number of arrangements.

In non-limiting examples, the 5′ CR may hybridize only to the 5′ adaptor, to the 5′ adaptor and the 5′ most portion of the SOI or only to the 5′ most portion of the SOI. Likewise, the 3′ CR may hybridize only to the 3′ adaptor, to the 3′ adaptor and the 3′ most portion of the SOI or only to the 3′ most portion of the SOI.

The 5′ CR may hybridize to a sequence wholly upstream of (ie more towards the 5′ end of the tethered polynucleotide than) the SOI, (eg such that its 5′ most nucleotide is complementary to a nucleotide upstream of the 5′ most end of the SOI), or the 5′ CR may hybridize to a sequence that overlaps the 5′ end of the SOI (eg such that the 5′ most nucleotide is complementary to a nucleotide within the SOI), or the 5′ CR may hybridize to a sequence wholly within the SOI, (eg such that the 3′ most nucleotide of the 5′ CR may hybridize to the 5′ most nucleotide of the SOI, or to a more 3′ nucleotide).

Likewise, the 3′ CR may hybridize to a sequence wholly downstream of (ie more towards the 3′ end of the tethered polynucleotide than) the SOI, (eg such that its 3′ most nucleotide is complementary to a nucleotide upstream of the 3′ most end of the sequence of interest), or the 3′ CR may hybridize to a sequence that overlaps the 3′ end of the SOI (eg such that the 3′ most nucleotide is complementary to a nucleotide within the SOI), or the 3′ CR may hybridize to a sequence wholly within the SOI, (e.g. the 5′ most nucleotide of the 3′ CR may hybridize to the 3′ most nucleotide of the SOI).

In some circumstances, the 3′CR may hybridize and extend, before the 5′ CR hybridizes to its target. In these cases the extending 3′CR may block hybridization of the 5′ CR and the amplification will fail. this can lead to lower yields of extended polynucleotide and poorer signal in sequencing reactions To prevent this from happening, the 5′ CR maybe caused to hybridize to the tethered polynucleotide before the 3′ CR is hybridized. This ensures that the 3′ end of the probe cannot be extended before the 5′ end of the probe is hybridized (see FIG. 4A). In one approach, the sequence of the 5′ and 3′ CRs are selected such that the Tₘ of the 5′ CR is higher than that of the 3′ CR. A protocol comprising an appropriate temperature profile will then insure the 3′ extension and ligation occurs as required. For some nucleotide sequences it may be possible to select sequences having an appropriate Tₘ and to which 5′ and 3′CRs of the MIP will hybridize in the above manner. In others it may be necessary to provide 5′ and 3′ adaptors comprising the sequences having an appropriate Tₘ. Such adaptors may be added to an polynucleotide or sequence of interest using standard methodologies at an appropriate point in any preparation protocol or workflows.

The Tins of the 5′ and 3′ CRs should differ sufficiently to ensure that the 5′CR hybridizes before the 3′CR, when an appropriate temperature profile is used. The profile will take into account speed of throughput, yield and undesired side products. Typically the Tins should differ by between 2 and 20° C. e.g. at least 2, 3, 4, 5, 6, 7, 8, 9, 10, 11, 12, 13, 14 or 15° C. or more, but an approximate 10° C. difference has been found to give good results.

In a further embodiment therefore the invention provides an improved molecular inversion probe for use in the preparation of a clonal cluster of tethered polynucleotides, such as this described further herein the molecular inversion probe comprising, or consisting of, in a 3′ to 5′ direction: (a) a 3′ CR, (b) an optional linker region; and (c) a 5′CR wherein the sequence of the 3′ CR is selected to hybridize to a first portion of a tethered polynucleotide and the sequence of the 5′ CR is selected to hybridize to a second portion of the tethered polynucleotide separate from the first portion; and wherein the Tm of the 5′ CR is higher than that of the 3′ CR. This approach is illustrated in FIG. 4B.

As detailed further above and elsewhere herein, the first portion of the tethered polynucleotide may be separated from the second portion of the tethered polynucleotide (that is to say, the two hybridization sites for the CRs of the MIP) by a sequence of interest, a complement of a sequence of interest, or a portion of them, depending on the choice of sequences to which the 5′ and 3′ CRs hybridize.

Step (b) of the process described above for the in situ formation of the circular probe may then include the step of hybridizing the 5′ CR of the probe to the tethered single stranded polynucleotide at a temperature above the Tₘ of the 3′ CR but at, or below, the Tₘ of the 5′ CR; and then hybridizing the 3′ CR of the probe to the polynucleotide at a temperature below the Tₘ of the 5′ CR, particularly at or below the Tₘ of the 3′CR.

An example MIP for use in this protocol is given below. This MIP was used in aspects of Example 6 as described further below and may be used with a temperature profile of 95° C. 2 mins, 55° C. 10 mins, to allow hybridization of the 5′ CR followed by 45° C. 30 mins. to allow hybridization of the 3′CR before extension and ligation.

SEQ ID NO: 1
/5Phos/ATGAAGCCAAGGCTGGTGGGTCGACAGCAGCTTCAACATTCGTTAGTCGAATCAG
TCCTGTCCGAGAAAACGAGACATGCC

In a further aspect of the invention is provided a method for preparing a clonal cluster of a tethered polynucleotide comprising: (i) providing a single stranded polynucleotide tethered to a surface through either its 5′ end; (ii) carrying out a first clonal amplification of the single stranded polynucleotide to provide a first cluster of polynucleotides tethered to the surface through their 5′ ends; and (iii) extending the tethered single stranded polynucleotides in the first cluster, by rolling circle amplification using a MIP as described herein and particularly the improved MIP described above. The tethered polynucleotide may be any of those described further elsewhere herein. In one approach, the method for the rolling circle amplification may comprise: (a) contacting the polynucleotides of the first cluster with a MIP, such as the improved MIP described above (b) hybridizing the 5′ CR of the MIP to the tethered single stranded polynucleotide at a temperature above the Tm of the 3′ CR but at, or below, the Tm of the 5′ CR; followed by hybridizing the 3′ CR of the probe to the polynucleotide at a temperature at or below the Tm of the 3′ CR; (c) optionally extending the 3′ end of the MIP; (c) cyclizing the probe by ligating the optionally extended 3′ end of the probe to the 5′ end of the probe; and (d) contacting the cyclized probe with a strand displacing polymerase to extend the single stranded polynucletide.

As already noted, the RCA approach to sequence extension can be applied whether the SOI has a known sequence, a partially known sequence or a completely unknown sequence. Where the 5′ and 3′ CRs hybridize only to sequences within the 5′ and 3′ adaptors of the SOI, then unknown sequences can be amplified. Other arrangements, where the adaptors hybridize at least partially to the SOI, generally require knowledge of the sequence of the SOI or at least part of it.

The linear probe may additionally comprise a linker region. The linker region, where present, extends from the 3′ end of the 5′ CR to the 5′ end of the 3′ CR. Likewise where the probe is provided as a preformed cyclic probe, it too may additionally comprise a linker region extending from the 3′ end of the sequence complementary to the SOI to the 5′ terminus of the sequence complementary to the SOI, or where the probe comprises regions complementary to any flanking regions present in the tethered polynucleotide 3′ end of the 3′ flanking region to the 5′ end of the 5′ flanking region, where present.

In either case, the linker may comprise one or more functional sequences, which will be reproduced in in complement as the linker is extended. These can include, for example, sequences complementary to sequencing primers or of amplification primers for example or may include sequences complementary to a further capture oligo as described elsewhere herein.

In one embodiment the linker comprises a sequence that, when duplicated in complement, hybridizes to a second species of capture oligo, bound to the surface. In some embodiments this sequence is different to those of the 3′ or 5′ adaptor and provides for the optional use of branching RCA in the second round of clonal amplification as described further elsewhere herein.

The length of the linker region need only be sufficient to accommodate any functional sequences required therein and to ensure that the probe cyclizes efficiently. Accordingly the linker may be any suitable length. For example, the linker may be 40-160 nt long, 40-70 nt is preferable. Shorter linkers ensure smaller circular probes and so less steric hindrance as well as more efficient RCA requiring fewer nucleotides.

In some embodiments, once hybridized to the tethered polynucleotide, the 3′ terminus of the linear probe may be separated from the 5′ terminus by one or more nucleotides. Typically the 3′ and 5′ termini are separated by all or part of the SOI, depending on the position of the sequences to which the 5′ and 3′ CRs hybridize. The 3′ terminus of the probe may be extended using a polymerase, preferably a polymerase lacking 5′ to 3′ exonuclease activity. Example polymerases include T4 polymerase, or Taq polymerase. Once the 3′ terminus is extended the extended 3′ terminus may be ligated to the 5′ phosphorylated terminus using a ligase, such as T4 ligase or Taq ligase in order to cyclize the probe. In some embodiments the 5′ and 3′ CRs of the linear probe, in combination, extend to cover the whole SOI, in which case, no extension is necessary and the 3′ and 5′ termini are simply ligated to cyclize the probe.

Whether the circular probe is provided preformed or is formed in situ, once the probe is in place the 3′ end of the tethered polynucleotide may be extended using the circular probe as a template. Adding one or more further copies of the SOI to the 3′ end of the tethered polynucleotide provides a cluster of tethered polynucleotides in which polynucleotides of the cluster (or at least a portion of them) comprise a plurality of copies of the nucleotide sequence of interest. Extending tethered single stranded polynucleotides in the first cluster, provides additional copies of the SOI without requiring increases in density of the capture oligo and without expanding the area occupied by the original cluster.

In some embodiments, for example where the circular probe does not hybridize to the full 3′ adapter of the SOI, a short single stranded 3′ overhang may be present, in which case this can be removed using a 3′-5′ exonuclease, or advantage can be taken of 3′-5′ exonuclease activity of a polymerase such as Phi29 polymerase.

In some cases, the 3′ end of the tethered polynucleotide may be extended around the probe before circularization and ligation is complete. If that occurs, the 5′ end of the probe may be displaced from the polynucleotide and circularization and ligation may not occur. Thus in some embodiments extension of the 3′ end of the polynucleotide is prevented (or at least delayed) whilst gap filling extension and ligation is completed. In some embodiments this is achieved by hybridizing the single stranded circular polynucleotide probe to the tethered polynucleotide leaving a 3′ single stranded overhang. Advantageously the overhang is not complementary to the probe, thus preventing formation of a double stranded feature suitable for extension by a polymerase. The 3′ extension can then be removed by an exonuclease activity before duplication of the circular probe.

In a further embodiment the single stranded circular polynucleotide probe is hybridized to the 3′ adaptor leaving a 3′ overhang sufficient to hybridize a protecting oligonucleotide whose 5′ end is co terminal with the 3′ end of the overhang. The oligo duplex has a Tm less than that of the probe, preventing strand extension until the oligo is removed by raising the temperature above that of the oligo duplex. The overhang can then be removed as before.

Extension of the 3′ end of the tethered polynucleotide around the probe is carried out using a polymerase, preferably by a strand displacing polymerase. In this way the probe is continuously separated from the extending amplicon as synthesis proceeds and multiple copies of the SOI are synthesized. In the extended amplicon copies of the SOI are interspersed with a sequence complementary to the rest of the circular probe. Depending on the sequence to which the 5′ and 3′ CRs of the linear probe hybridize, the SOI may also be flanked by full length or truncated forms of the adaptors.

If the CRs incorporate complementary copies of the various functional sequences in the adaptor the functional sequence will be reproduced in the extending amplicon and the functional sequences will remain associated with the SOI. In some embodiments therefore, the sequence of the 5′ CR of the probe and of the 3′ CR of the probe are selected such that, following optional extension and ligation, a single stranded circular probe is provided comprising a complementary copy of the sequence of interest and a copy of either the 5′ adaptor, the 3′ adaptor or both; or sufficient of either adaptor to include one or more functional sequences such as any of those discussed further herein. It is particularly preferred that where the 3′ adaptor comprises a region complementary to a sequencing primer, the sequence of the 3′ CR is selected such as to preserve the functional relationship between the sequencing primer and the SOI as described further herein.

The combination of MIP hybridization, circularisation and rolling circle amplification may be referred to herein as Linear Cluster Concatemerization or LCC.

In one embodiment a second species of capture oligo may be provided on the surface as a mixed population with the first capture oligo. The second capture oligo hybridizes to the complement of a sequence within the linker region of the circular probe, but does not hybridize to the SOI or to its 5′ or 3′ adaptors. As the circular probe is repeatedly duplicated it provides complementary copies of the linker interspersed with copies of the SOI and adaptors. The complementary copies of the linker expose multiple sequences that hybridize to the second species of capture oligo and so the extending strand is captured at multiple points by the second capture oligos. The 3′ end of the capture oligo is then extended by the polymerase to form a complementary copy of the SOI and sense copies of the linker. As the new strand extends it displaces the next duplex from the next copy of the second capture oligo, producing multiple tethered polynucleotides comprising a plurality of complementary copies of the SOI alternating with copies of the linking region. It is then possible to sequence both forward and reverse strands of the SOI from the same cluster using sequencing primers complementary to the appropriate adaptor.

Once the extended clonal cluster has been provided it may be used for a variety of downstream processes as described further herein. In one embodiment, the sequence or partial sequence of the SOI within the 3-D cluster may be determined by a method of nucleotide sequencing.

Sequence determination may be by any method appropriate to a polynucleotide immobilized or tethered to a surface, particularly the approach known as “sequencing by synthesis (SBS)”. In this approach, following preparation of the clonal cluster, a sequencing primer is annealed to the 3′ adaptor and the incorporation of successive nucleotides into the extending strand, complementary to the SOI, is detected. In some approaches the nucleotide is labelled with a fluorescent dye. The dye may act as a strand synthesis terminator. After each dNTP incorporation, the dye is imaged to identify the base and cleaved to allow incorporation of the next nucleotide. In other approaches the clonally amplified polynucleotides are tethered to the surface of a semiconductor chip, such as an ISFET and incorporation of each nucleotide in the SOI is detected by detecting protons released (1 per nucleotide incorporated) during the incorporation of the dNTP into the sequence. In this case dNTPs need not be fluorescently labelled.

There are a variety of variations of next generation sequencing including those that rely on SBS but the clonal amplification approach described herein is suitable for use with any that may be carried out on immobilized populations of polynucleotides.

In a further embodiment, the invention also provides a substrate having coupled to its surface a plurality of single stranded polynucleotides arranged as a plurality of clonal clusters, wherein the polynucleotides within each clonal cluster comprise a plurality of alternating copies of a first nucleotide sequence and a second nucleotide sequence, wherein the first nucleotide sequence is common to the single stranded polynucleotides of each cluster and wherein the first sequence is not. In a further embodiment the invention also provides a clonal cluster of single stranded polynucleotides as described herein wherein polynucleotides of the cluster are tethered to a surface through their 5′ ends.

In some embodiments, the first sequence, equivalent to the sequence of interest as discussed further elsewhere herein, may be present within the cluster in sense or antisense, but not both. In some embodiments, both sense and antisense versions of the first sequence are present in the cluster. In this case the polynucleotides of each cluster comprise a sense and antisense copy of the first sequence and a sense and antisense copy of the second sequence, the sense and antisense copies of the second sequence being common to the single stranded nucleotides of each cluster, but the sense and anti-sense copies of the first sequence are not.

Typically the polynucleotides within each cluster comprise multiple copies of the first sequence (or its antisense). In preferred embodiments they comprise at least 2, preferably at least 3, more preferably at least 4, and even more preferably at least 5 copies of the first sequence or its antisense. Likewise the polynucleotides within each cluster comprise multiple copies of the second sequence (or its antisense). In preferred embodiments they comprise at least 2, preferably at least 3, more preferably at least 4, and even more preferably at least 5 copies of the second sequence or its antisense.

Typically polynucleotides within each cluster have free 3′ ends. Typically, since the surface initially comprises 1, 2, 3, 4, 5 or more species of capture oligo, in some instances areas that do not comprise clustered polynucleotides comprise unused capture oligos and may therefore also comprise 1, 2, 3, 4, 5 or more species of capture oligo which typically remain un hybridized to a polynucleotide. Typically however, within clusters, unused capture oligos are rare since they become exhausted locally during the first round of clonal amplification. In some embodiments the surface comprises a single species of capture oligo, in some embodiments the surface comprises 2 species of capture oligo.

The number of clusters containing the same first sequence is dependent on the number of unique SOIs present. Thus, for example, if only 2 SOIs are present 50% of the clusters will contain the same 1st sequence.

Herein a free 3′ end means that the terminal 3′ nucleotide has a free 3′—OH group capable of being extended by a polymerase, such as a strand displacing polymerase. It is not part of a hairpin or other secondary structure.

Herein clonal amplification is the multiplication of a single polynucleotide to provide a plurality of copies (clones) having the same sequence. The single polynucleotide and its clones are tethered to the surface of a substrate typically through their 5′ or 3′ terminal nucleotides, preferably through the 5′ terminal nucleotides. A clonal cluster is a plurality of polynucleotide clones grouped together in a discrete region of the surface, particularly in a closely packed manner. Clonal clusters are typically derived from clonal amplification of a single originating tethered polynucleotide, but they may alternatively be derived by spotting a composition comprising a single species of polynucleotide onto a surface. In some embodiments clonal clusters may intentionally comprise more than one species of polynucleotide. For example, they may comprise copies of a single stranded polynucleotide and complementary copies of the single stranded polynucleotide. Clonal clusters may overlap with other clonal clusters, but it is preferred that they do not.

Herein two sequences are considered complementary if each nucleotide in one strand undergoes sequential Watson Crick base pairing with a nucleotide in the other strand. The two strands are complementary copies of each other.

Herein, where two sequences are said to hybridize to each other, they do so under the conditions of the process in question, thus where a primer is said to hybridize to an nucleotide sequence it does so under the conditions (ionic strength, pH, temperature for example) of primer extension, sequencing and so on.

Herein the term “polynucleotide” means a polymer of two or more nucleotides joined by covalent linkages, typically phosphodiester linkages. In some cases the polynucleotide may include “non-natural” linkages such as phosphorothioate linkages, for example where it is advantageous to reduce or prevent exonuclease activity. The term polynucleotide fragment, oligonucleotide or oligo has the same meaning as polynucleotide and the terms are used interchangeably. Polynucleotides may be single stranded or double stranded and may be DNA, RNA or a hybrid DNA/RNA duplex.

Herein the term “tethered” means that the polynucleotide referred to is bound to the surface or to any coating applied thereto through a chemical bond, preferably covalently.

### FIGURES

FIG. 1 provides an illustrative outline schematic of clonal amplification using the recombinase polymerase approach. A is a double stranded DNA fragment; B is a fragment having 5′ and 3′ adaptors; C is a melted duplex; D illustrates the process of clonal amplification using recombinase polymerase amplification; and E is a substrate having a tethered cluster of identical sequences.

FIG. 2 provides an illustrative schematic showing one approach to rolling circle amplification of the sequence of interest, particularly applicable to the case where the sequence is known.

FIG. 3 provides a further illustrative schematic showing one approach to rolling circle amplification of the sequence of interest using molecular inversion probes (MIPs).

FIG. 4A illustrates the effect of the 3′CR binding prior to the 5′CR. (i) illustrates a successful circularization and (ii), illustrates an unsuccessful circularization.

FIG. 4B illustrates a modified MIP in which the 5′CR has a Tₘ of 60° C. and the 3′ CR has a Tₘ of 55° C. An example circularization protocol to be used may then be: Denature—95° C. for 2 minutes. 5′ annealing—60° C. for 5 minutes, 3′ annealing, extension and ligation—50° C. for 10 minutes.

FIG. 5A is a fluorescence micrograph composite image showing the results of a spotting trial of clonal amplification at 50 uM capture oligo (optimal for RPA) and 2 uM capture oligo (optimal for RPA+LCC) on both YdfU template and ddl template. Test templates were clonally amplified by either RCA only or by RCA followed by LCC. Templates were tethered to the surface of an ISFET. Individual clonal clusters are just visible in some spots at this magnification.

FIG. 5B shows SensoSpot® scan data of the spots in 5A.

FIG. 6 illustrates data from sequencing reactions carried out on RPA clonal clusters (YdfU) and dual template RPA+LCC clonal clusters (YdfU and ddl). Graphs A provide read length data for RPA and RPA+LCC clusters. Graphs B give the distributions of ARL-e.

In Graphs A individual reads are plotted as Aligned Read Length (ARL; total alignment length from start to end positions) on the x-axis versus Aligned Read Length-Errors (ARL-e; total alignment length from start to end positions minus the number of errors on the y axis). The histogram sections indicate the distribution of reads on the opposite axis. The histogram across the top indicates the distribution of ARL (bp); the histogram on the right-hand side of the plot indicate the distribution of ARL-e (bp). Graphs B give the distributions of ARL-e. Graphs C show the signal derived from incorporation of a single nucleotide into the extending sequencing strand during Sequencing by Synthesis (RPA and LCC 1 mers distinguished from each other).

FIG. 7 show fluorescence photomicrographs illustrating clonal clusters derived from single captured polynucleotides on a flood filled ISFET chip. Clusters derived by RPA (A) are visible as pale cloud-like features (arrowed). The white dots are control spots. Micrograph B shows clonal clusters resulting from RPA followed by LCC amplification. (arrowed). RPA was carried out on capture oligos applied at 25 uM, whilst the RPA+LCC was carried out on oligos applied at 1 uM. The capture oligo was extHDA72R and the template was YdfU.

The graphs (C) illustrate the intensity of individual pixels across each image for 5 chip images. The intensity (X axis) is plotted against the % of pixels of at least that intensity (Y) for each chip.

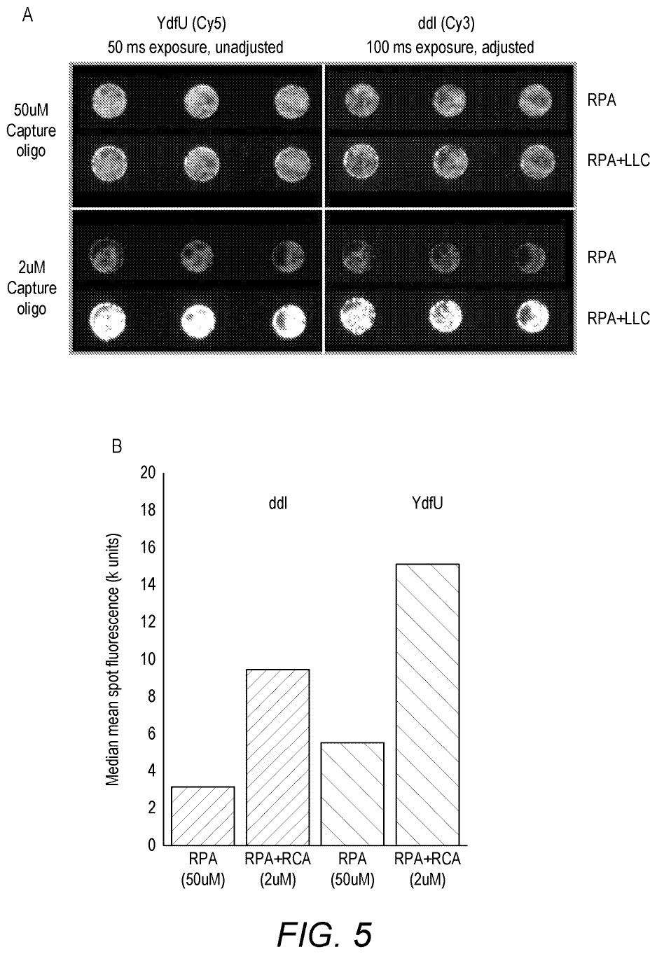

FIG. 8 shows data from a similar experiment to FIG. 5 but in which the improved MIP is used. The graph as per FIG. 7, illustrates the intensity of individual pixels across each image for 6 spot images. The intensity (X axis) is plotted against the % of pixels of at least that intensity (Y) for each chip.

FIG. 9: provides grey scale images of a single spot resulting from tethering of the capture oligo onto a glass slide, followed by hybridization of an equimolar mixture of YdfU and ddl templates and subsequent clonal amplification. The spot was probed with both YdfU and ddl fluorescent probes after the RPA stage and then after LCC. In the left hand images arrows indicates individual ddl clonal clusters. The right hand images highlight the same clones as dark spots illustrating that these clusters are not detected by the YdfU probe even after LCC and clonality is maintained.

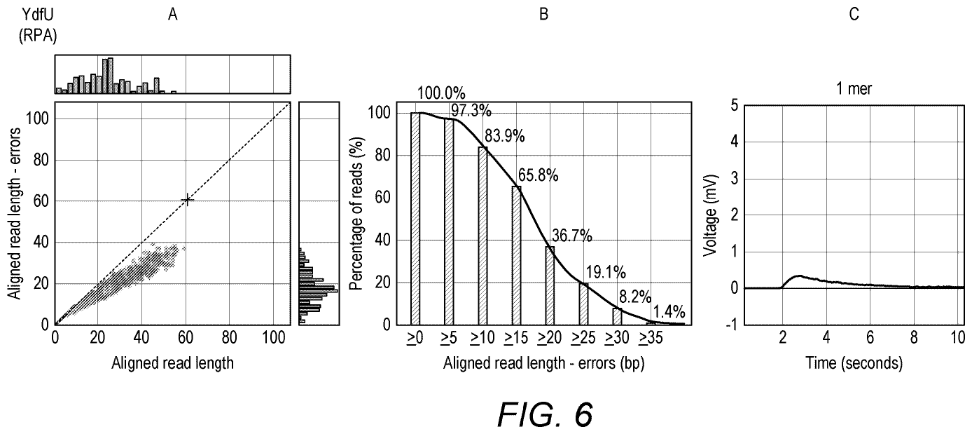

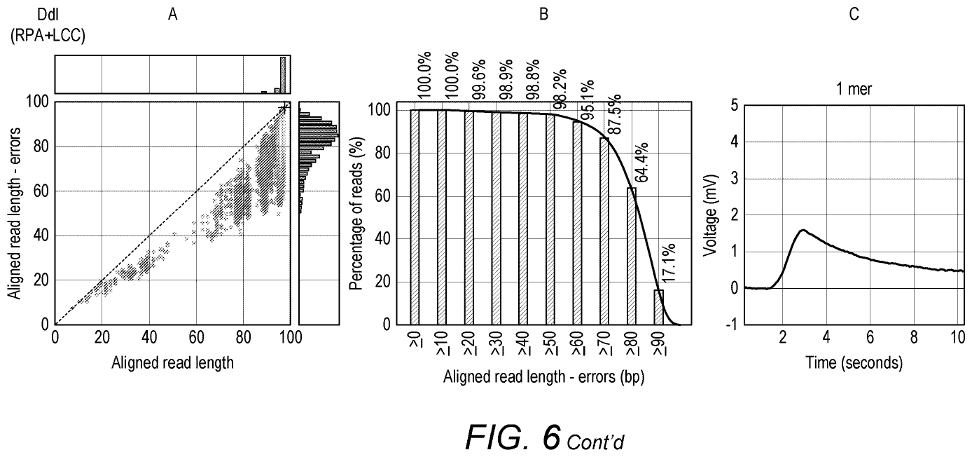

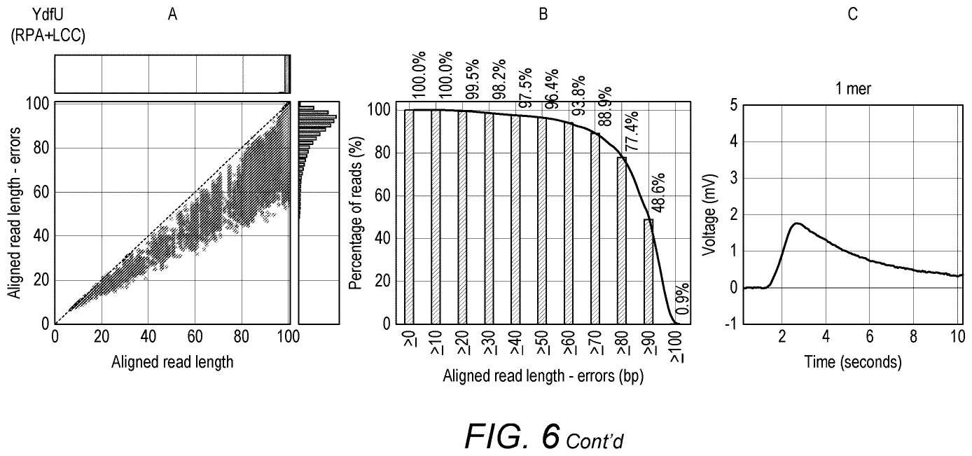

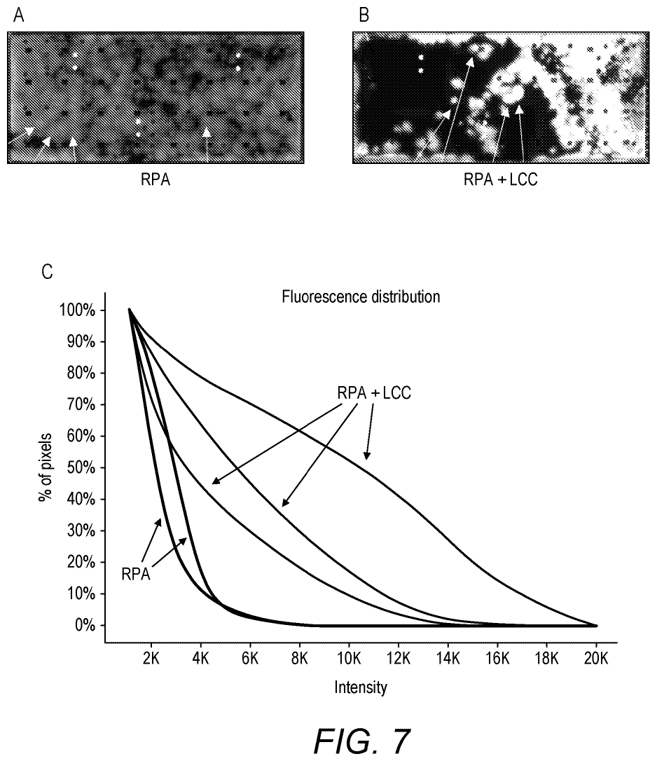

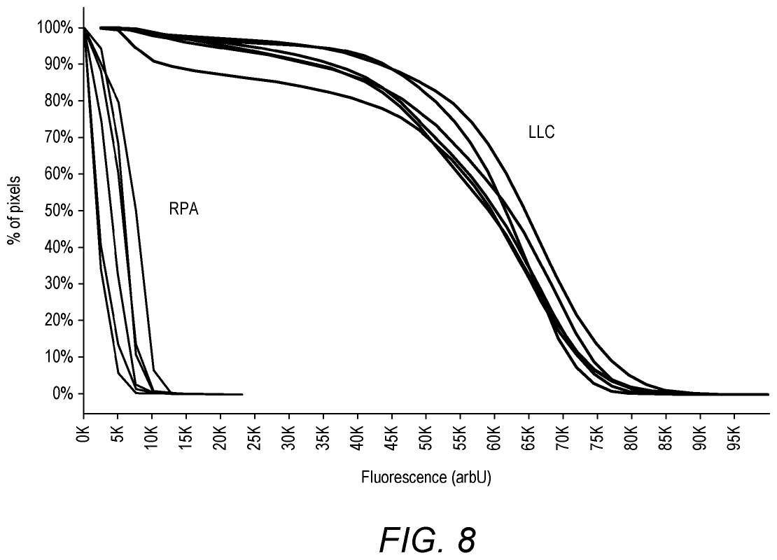

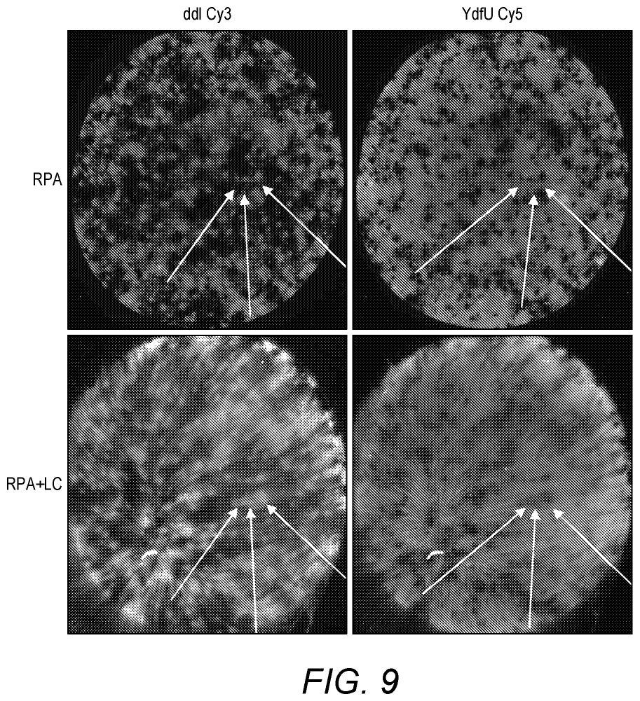

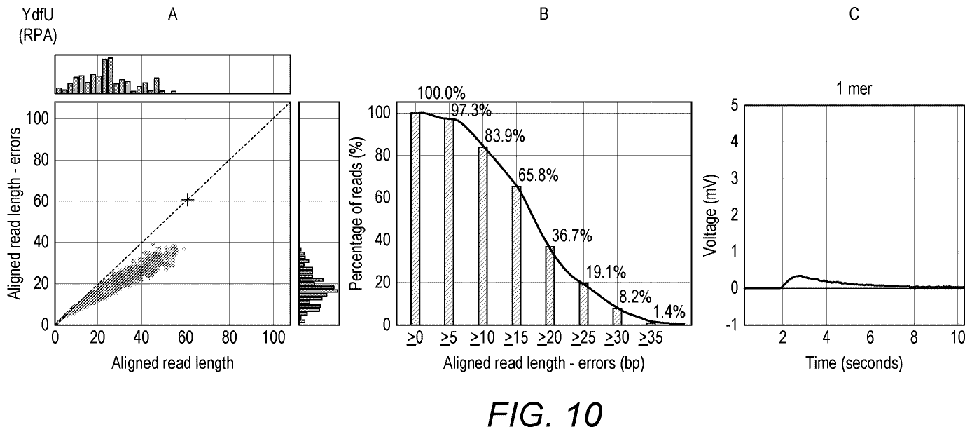

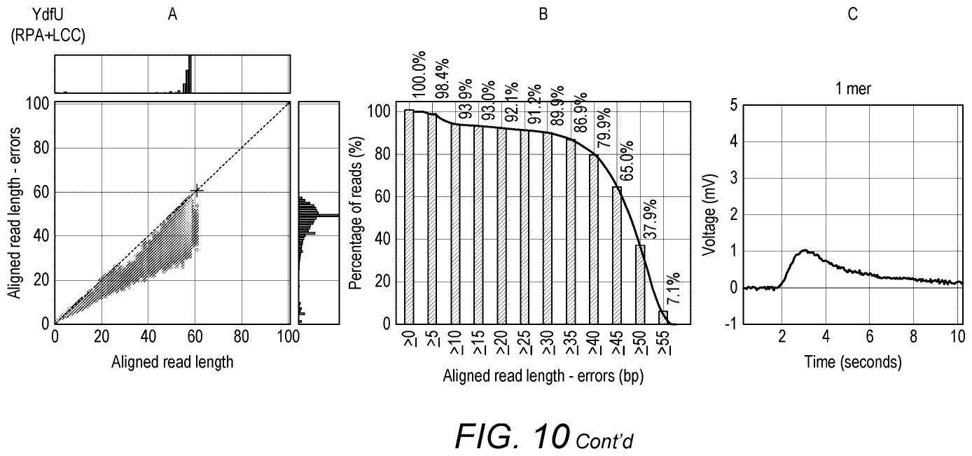

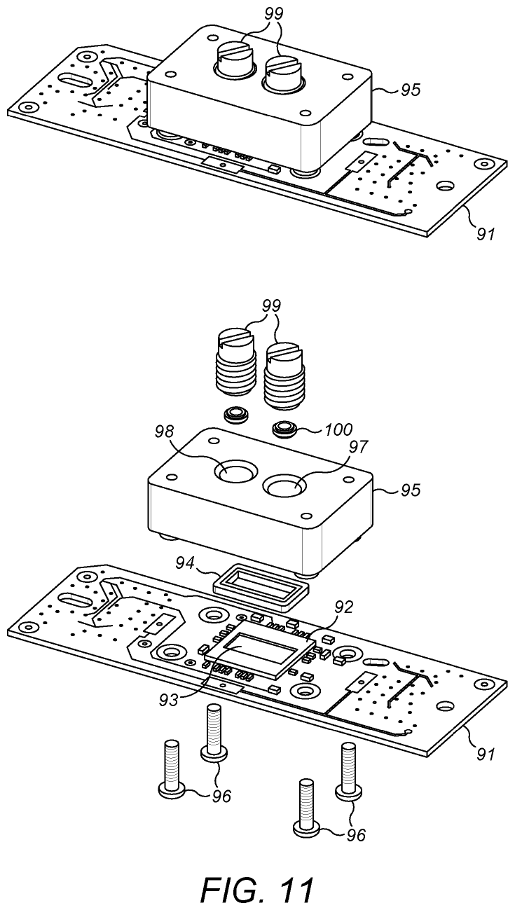

FIG. 10: illustrates 1 mer intensity data from a sequencing reactions carried out on YdfU clonal clusters derived from RPA only or RPA+LCC in which the circles are generated in solution. Graphs A provide read length data for clusters generated by RPA and by RPA+LCC using circles generated in solution. Graphs B give the distributions of ARL-e as per FIG. 6.

Graphs C show the signal derived from incorporation of a single nucleotide into the extending sequencing strand.

FIG. 11 illustrates a custom flow cell attached to the ISFET chip to assist delivery and flow of reagents onto the ISFET chip surface.

### DISCUSSION OF THE FIGURES

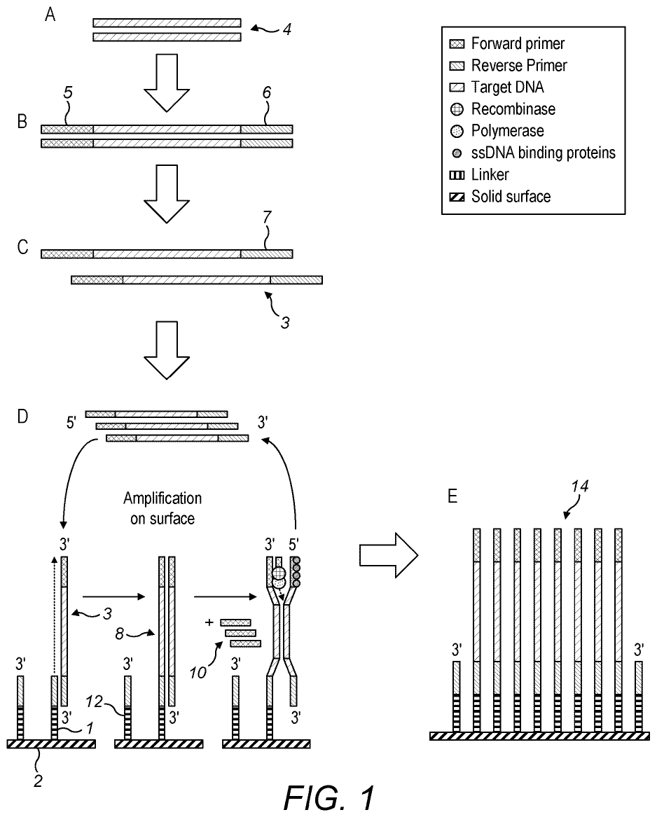

With reference to FIG. 1, and descriptions elsewhere herein, clonal amplification using surface phase recombinase polymerase amplification RPA uses a reverse primer (capture oligo) (1) bound to a surface (2) to capture a single stranded polynucleotide (3). The process of tethering the capture oligo is described elsewhere herein and the generality of the process is well known.

The single stranded polynucleotide (3) may be derived from an example upstream workflow (A, B, C), which can include provision of a polynucleotide fragment (4) and the provision of adaptors (5, 6) at the 5′ and/or 3′ ends. Separation of the duplex (if the fragment is double stranded) provides a single stranded polynucleotide (3).

The capture oligo (1) bears a sequence in the 3′ region, which is complementary to the 5′ region of the polynucleotide, such as the 5′ adaptor (7) and this facilitates capture of the polynucleotide. The 3′ end of the capture oligo is extended by a polymerase along the captured polynucleotide to provide a complementary copy of the polynucleotide. This complementary copy (8) is tethered to the substrate through its 5′ end which is now an extension of the original capture oligo (1). Concerted action of recombinase and ssDNA binding proteins allows access of solution phase forward primers (10) complementary to the 3′ end of the tethered complementary copy, to the duplex. A strand displacing polymerase then extends the hybridised forward primers over the complementary strand and displaces the original oligonucleotide whilst providing a duplicate copy of it. The original polynucleotide (3) is then free to be captured by a further local capture oligo (12). Repeated rounds of primer hybridization and extension provide multiple copies of the original polynucleotide which themselves are released into solution and captured by local capture oligos thus providing templates for further rounds of amplification. After denaturing the remaining duplex molecules, the tethered oligo nucleotides in the clonal cluster are clonal copies of the original polynucleotide, but in complement (14).

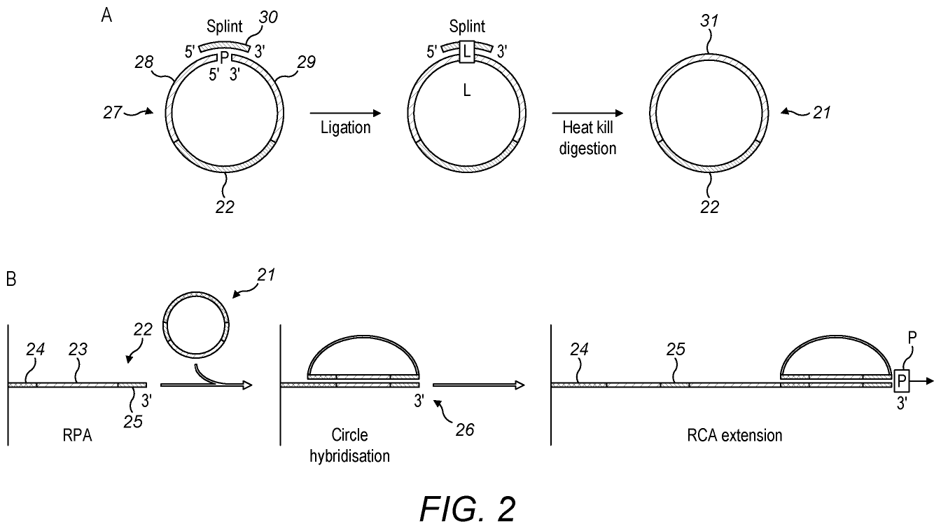

With reference to FIG. 2, in some approaches, particularly where the SOI is known, the circular probe can be provided as a preformed circular probe (21), comprising a complementary copy of the sequence of interest (22). The complementary copy of the SOI may then be hybridized to the tethered SOI (23), (and optionally to any 5′ or 3′ adaptors (24, 25) and the 3′ end of the tethered oligonucleotide (26) is then extended by repeated duplication of the circular probe in complement using a strand displacing polymerase (P). The circular probe can be prepared from a linear probe (27) comprising a complementary copy of the sequence of interest (22). One or more flanking regions (28, 29) may be included. The 5′ and 3′ ends of the linear probe are brought together by a splint oligo (30) which is complementary to the two ends, and ligated (L). In the approach illustrated, the optional flanking regions are brought together to form a linker (31). If necessary, separation of the duplex and digestion of the splint leaves the circular probe intact. If the flanking regions comprise complementary copies of adaptors, these will be incorporated into the extending amplicon, even if not present on the tethered polynucleotide. In practice the link between the free ends of the circle can occur at any point in the circle and does not need to be two flanking regions, see example 8.

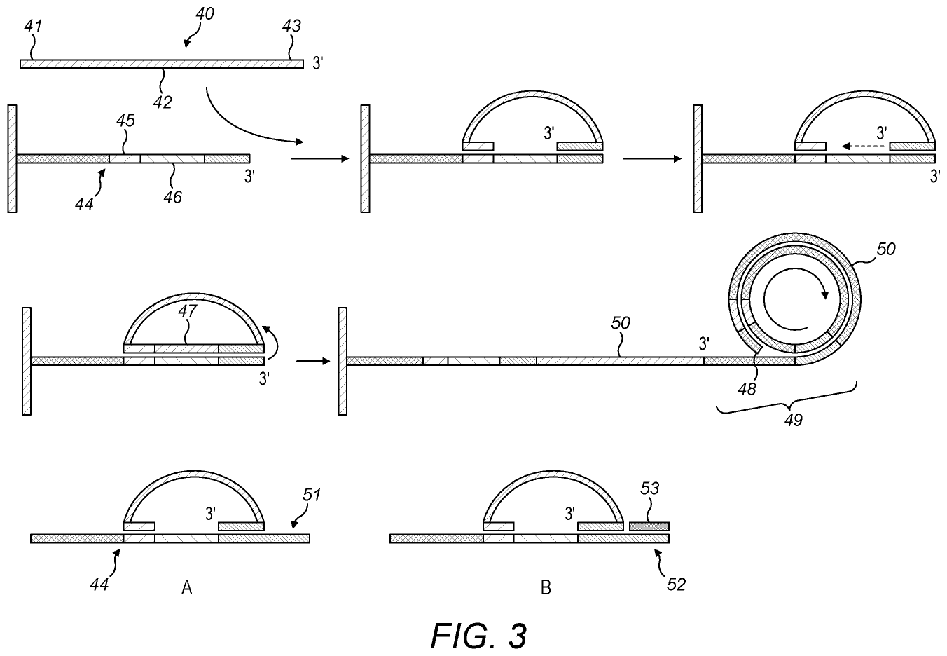

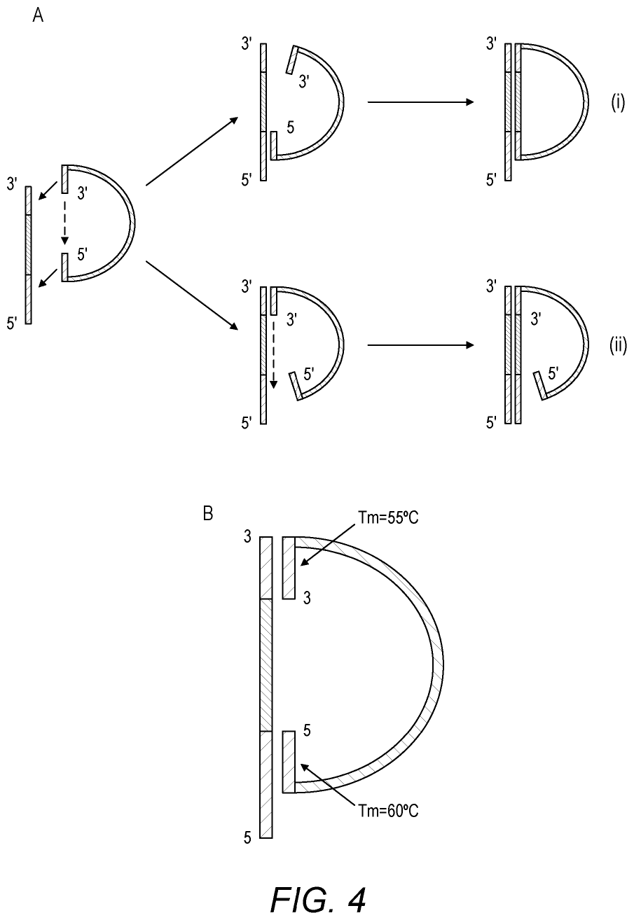

With reference to FIG. 3, in one approach, the circular probe is provided as a linear, single stranded polynucleotide (40) comprising in a 5′ to 3′ direction, a 5′ CR (41), a linker region (42) and a 3′ CR (43).

The CRs hybridize to separate sequences on the tethered polynucleotide (44). In this case they hybridize to complementary sequences within the 5′ and 3′ adaptors (45, 46). The 3′ end of the linear probe is extended using a DNA polymerase providing a complementary copy of the SOI (47) using the tethered polynucleotide as a template. The probe is then cyclized by ligating the extended 3′ end of the probe to the 5′ end of the probe. The 3′ end of the tethered polynucleotide template is extended around the probe using a strand displacing polymerase, which ultimately displaces the 3′ end of the duplex (48), as it extends the amplicon.

Extension of the amplicon using RCA provides a plurality of duplicates of the tethered polynucleotide template (49) separated from each other by complementary copies of the linker (50). The 3′ end of the original tethered polynucleotide is extended by repeating copies of the circular probe in complement.

A and B illustrate approaches to preventing extension of the 3′ end of the tethered polynucleotide outstripping the extension of the 3′ end of the MIP and therefore displacing the MIP from the template. In A, the 3′ CR of the MIP is hybridized to the tethered polynucleotide (44) leaving a 3′ overhang (51) which is not complementary to the MIP. This prevents formation of a double stranded feature having a free 3′ end suitable for extension by a polymerase. The 3′ extension can then be removed by 3′-5′ exonuclease activity before duplication of the circular probe. In B a 3′ overhang (52) is sufficient to hybridize a short protecting oligonucleotide (53) whose 5′ end is co terminal with the 3′ end of the overhang. The short duplex has a Tₘ less than that of the probe, preventing strand extension until the oligo is removed by raising the temperature above the Tₘ. The overhang can then be removed as before.

Referring to FIG. 11 a circuit board (91) comprises an ISFET chip (92), with a surface (93). A gasket (94) provides a seal between the chip (92) and a flow cell (95) which is secured over the ISFET chip (92) using screws (96) to provide a flow path over the chip, between an inlet (97) and an outlet (98). Inlet and outlet may be sealed by screw closures (99) sealed with an “O” ring (100) to prevent evaporation.

Other figures are discussed further in the appropriate example. The invention will now be illustrated by non-limiting examples and figures. Further embodiments of the invention will be apparent to the skilled person in light of these.

### EXAMPLES

The following buffers and reaction mixes were used in the examples below unless stated otherwise:

TABLE 1
RPA Buffer Mix supplied as a premix. This mix contains
all non-enzymatic reagents of the final RPA mix.
RPA Buffer Mix

| | Stock | | Final | | Volume/ |
|---|---|---|---|---|---|
| Reagent | Concentration | | Concentration | | rxn (ul) |
| Carbowax 20M | 20% | | 6% | | 12.00 |
| DTT | 100 | mM | 5 | mM | 2.00 |
| Uvs reaction buffer | 20 | X | 1 | X | 2.00 |
| HDA72F (Table 1) | 100 | uM | 0.6 | uM | 0.24 |
| dNTPs mix (Each) | 25 | mM | 0.46 | mM | 0.74 |
| ATP | 100 | mM | 4 | mM | 1.60 |
| Phosphocreatine | 250 | mM | 12 | mM | 1.92 |

| | | | |
|---|---|---|---|
| Water | N/A | N/A | 6.22 |

| | |
|---|---|
| Total Volume | 26.72 |

TABLE 2
RPA Core mix - supplied as a premix. This mix contains
all enzymatic reagents of the final RPA mix.
RPA Core Mix

| | Stock | | Final | | Volume/ |
|---|---|---|---|---|---|
| Reagent | Concentration | | Concentration | | rxn (ul) |
| T4 Gene 32 Protein | 10,000 | ng/ul | 250 | ng/ul | 1.00 |
| T4 UvsX DNA | 5,000 | ng/ul | 250 | ng/ul | 2.00 |
| Recombinase | | | | | |
| T4 UvsY Protein | 2,000 | ng/ul | 100 | ng/ul | 2.00 |
| Creatine Kinase | 1,000 | ng/ul | 60 | ng/ul | 2.40 |
| Bsu DNA Polymerase, | 50 | U/ul | 0.75 | U/ul | 0.60 |
| Large Fragment | | | | | |

| | |
|---|---|
| Total Volume | 8.00 |

TABLE 3
RPA Final mix. This mix is a combination of
both RPA premixes and contains all reagents
required for surface phase RPA.
RPA Final Mix

| | Stock | Final | Volume/ |
|---|---|---|---|
| Reagent | Concentration | Concentration | rxn (ul) |
| Water | | | 5.28 |
| RPA Buffer Mix | 1.5 x | 1 x | 26.72 |
| RPA Core Mix | 5.0 x | 1 x | 8.00 |

| | |
|---|---|
| Total Volume | 40.00 |

Table 4: Circularization Mix. This mix contains all reagents required for MIP extension and GC ligation.

TABLE 5
RCA mix. This mix contains all reagents
required for the rolling circle amplification of
surface conjugated, RPA-generated clusters.
RCA Mix

| | Stock | | Final | | Volume/ |
|---|---|---|---|---|---|
| Reagent | Concentration | | Concentration | | rxn (ul) |
| Water | N/A | | N/A | | 33.00 |
| EquiPhi Buffer | 10 | x | 1 | x | 4.00 |
| dNTPs | 25 | mM | 1 | mM | 1.60 |
| DTT | 100 | mM | 1 | mM | 0.40 |
| EquiPhi29 DNA | 10 | U/ul | 0.25 | U/ul | 1.00 |
| Polymerase | | | | | |

| | |
|---|---|
| Total Volume | 40.00 |

### Example 1: Coupling Capture Oligos to a Surface

The semi-conductor chip utilized in this example was fabricated using standard CMOS methods and comprises an array of Ion-Sensitive Field-Effector Transistor (ISFET) sensors whose voltage output responds to changes in pH in a fluidic solution residing in wells above the IC. The wells are micron-sized and were produced by standard photo etching processes. A custom flow cell apparatus (see FIG. 9) was mounted on top of the IC and well assembly to facilitate the delivery of fluids over the surface of the chip. Oligonucleotides were coupled to the surface of the wells by first activating the well surface with an acrylamide-based polymer coating bearing free azide groups (MCP-Click™—Lucident Polymers, Sunnyvale, California, USA), followed by flooding the surface with a solution of the appropriate 5′ DBCO capture oligo at a concentration optimised for either 2-D, RPA approaches (50 uM) or for 3-D, (RPA+LCC), approaches (2 uM). The modified oligos were covalently coupled using standard dibenzocyclooctyl (DBCO)/azide click chemistry. The sequence of extHDA72R capture oligo is given below:

(SEQ ID NO. 2)
/5′DBCOTEG/AAAAAAACTCCTCTGGCACCGTGCTGCCTTGGCTTCATTGTG*G*T*
C

DBCOTEG = 5′ Dibenzocyclooctyl: Triethylene glycol. * = phosphorothioate bond (Integrated DNA Technologies-IDT-Iowa USA).

In an alternative approach, rather than flooding the chip, capture oligos were spotted onto pre cleaned chip surfaces in volumes of 280-300 pico-litres. The oligos were coupled onto the surface in the same manner.

In some experiments, glass slides were used instead of ISFET chips. Where glass slides were used as the substrate, these where first coated with Ta₂O₅ using e-beam evaporation according standard methods and subsequently coated with acrylamide-based polymer coating bearing free azide groups as described above, before conjugating the capture oligos.

### Example 2: Hybridization of Test Templates to Capture Oligos

Synthetic DNA oligos (purchased from IDT Technologies) representing the D-alanine-D-alanine ligase gene from Enterococcus faecalis (ddl) and the Qin prophage protein YdfU gene from E. coli (YdfU) were used as test templates. Their sequences are given below:

ddl template
(SEQ ID NO. 3)
AAAACGAGACATGCCGAGCATCCGCCGCGCTTCAATTCCTTGTTACTGATAGGCTGTTG
CTAAAGCATTTTGCAGCTCTTCTCGGTTTGACCACAATGAAGCCAAGGCAGCACGGTGC
CAGAGGAGTTTTTTT
ydfU template
(SEQ ID NO. 4)
AAAACGAGACATGCCGAGCATCCGCTGCGGGTATTACTTAGACCTGTTCTGGTG
CCTGAGCTTGGGCTGGTGGTCCTTAAGCCGGGCCGTGAATCCATACAGATAGAC
CACAATGAAGCCAAGGCAGCACGGTGCCAGAGGAGTTTTTTT

Underlined sequences are universal forward and reverse primer binding sites: Bold sequences are universal binding sites for MIP 5′ and 3′ CRs.

YdfU and ddl equimolar mix (2.5e4 copies/ul)−40 ul in 1× annealing buffer (20 mM Tris-HCl pH 7.5, 150 mM sodium chloride, 5 mM magnesium acetate, 0.01% v/v Tween 20 and 5% v/v DMSO) was hybridized to surface-conjugated extHDA72R oligos (conjugated as spots or floods) through the universal adapter regions by incubation at 95° C. for 2 minutes, 50° C. for 5 minutes and 20° C. for 10 minutes. Following hybridization, the surface was washed twice with 60 ul of 1×RPA wash buffer (0.06% SSC pH 7, 0.06% v/v Tween20).

### Example 3: Clonal Amplification of Captured Test Templates by RPA

Clonal amplification was performed by surface phase recombinase polymerase amplification (RPA). The captured test templates were incubated with 40 ul RPA Final Mix (Table 3) at 43° C. for 1 hour followed by 75° C. for 10 minutes before washing twice with 60 ul water. Following amplification, oligo duplexes were melted by incubation with 40 ul of 40 mM NaOH at 20° C. for 10 minutes and washed twice with 1×RPA wash buffer (0.06% SSC, 0.06% v/v Tween20).

### Example 4: Visualisation of Clonal Clusters

Initial clonal clusters amplified in the plane of the surface (2-dimensional clusters) were verified by annealing fluorescent oligos directed to specific sequences within the surface-tethered templates.

The clonally amplified, surface-conjugated templates were incubated with an equimolar mix of YdfU (Cy5) and ddl (Cy3) specific probes at 5 uM in 1× annealing buffer (20 mM Tris-HCl pH 7.5, 150 mM Sodium Chloride, 5 mM magnesium acetate, 0.01% v/v Tween 20 and 5% v/v DMSO) at 95° C. for 2 minutes, 50° C. for 5 minutes and 20° C. for 10 minutes. Following hybridization, the surface was washed twice with 1×RPA wash buffer (0.06% SSC, 0.06% v/v Tween20). The sequence of these probes is given below:

ddl/Cy3:
(SEQ ID NO. 5)
5′-Cy3-CAACGATTGCTCGAGAATCAT-3′
YdfU/Cy5:
(SEQ ID NO. 6)
5′-Cy5-TGCGGGTATTACTTAGACCTGTTC-3′
5′-Cy3 = 5′-terminal Cy3 fluorophore; 5′-Cy5 = 5′-terminal Cy5 fluorophore

Annealed fluorescent probes were visualised by imaging with a Sensospot® microarray scanner (Sensovation AG) using the red excitation filter for the YdfU Cy5 probe and the green excitation filter for the ddl Cy3 probe, both with a 10 ms exposure time.

### Example 5: Sequencing

### (a) Buffer/Reagent Preparation

Sequencing Solution: (7.5 mM MgCl2, 200 mM NaCl, 0.02% Tergitol NP-9) was prepared in a glass bottle using deionized water (18MΩ; Merck Millipore), 1M magnesium chloride solution, 5M NaCl and Tergitol™ NP-9 (neat; Merck). The MgCl₂ and NaCl stock solutions were added to the deionized water to yield final concentrations of 7.5 and 200 mM respectively. The solution was mixed to homogeneity using a magnetic stirring plate. To ensure dissolved carbon dioxide was removed, the solution was then sparged with nitrogen and maintained under nitrogen. Tergitol NP-9 (250 μl) was added to a final concentration of 0.02%.

Nucleotides: A 10 μM solution of each of dGTP, dCTP, dATP and dTTP was prepared by adding 12.5 μL of 100 mM stock of the selected nucleotide (Fisher Scientific, 11843933) to 125 mL of sequencing solution and adjusting to pH 8.05±0.01 using 10 mM NaOH. All dNTP solutions were prepared in a CO₂ free, nitrogen-controlled environment.

Wash solution was prepared by adjusting the pH of sequencing solution to 8.05±0.01 with 10 mM NaOH in a CO₂ free, nitrogen-controlled environment.

Annealing buffer (1×) was prepared by diluting a 20× stock of saline sodium citrate buffer (Life Technologies) to a final 1× concentration of 150 mM NaCl and 15 mM sodium citrate using Molecular Grade Water (Sigma).

Sequencing primer: A 5 μM working solution of sequencing primer (fluorescent probes from example 4 were used as sequencing primers) was prepared by adding 1.25 μL sequencing primer (100 μM stock) to 5 μL of 1× annealing buffer to give a final composition of 5 μM sequencing primer in 0.8× annealing buffer.

Polymerase: A 25.4 U/μL working stock of sequencing polymerase was prepared by diluting 1 μL of IsoPol BST+ DNA Polymerase at 2 kU/μL (ArcticZymes) in 79 μL of sequencing solution and thoroughly mixed.

### (b) General Sequencing Protocol

The ISFET chip was provided with a custom flow cell arrangement (FIG. 11), in which the surface of the ISFET was exposed to solutions flowing through the cell. This allowed easy delivery and control of reagents flowing over the surface. The flow cell was flushed twice with 1× ThermoPol Buffer [4.5 mL 10× ThermoPol buffer (New England Biolabs), 27 μL Tween 20 (100% stock, Merck Life Science, P9416), 40.5 mL Molecular Grade Water (Sigma)].

Fluorescent probes applied according to example 4 as part of the workflow, acted as sequencing primers. Sequencing polymerase (25 U/μL) was loaded into the flow cell and incubated for 10 minutes at ambient temperature (about 20-26° C.). The flow cell was flushed with 200 μL 1× ThermoPol Buffer.

A priming step was performed in which wash solution was flowed across the chip at 5 mL/min. An electrical response test was performed by biasing the reference electrode with increasing voltage steps. The resulting change in mV output measured by the ISFETs on the IC was used to determine the relationship between reference electrode potential and the corresponding potential seen on the ISFETs, which then was used to determine the optimal reference electrode potential for the experiment.

Sequencing was then performed cycle by cycle. During each sequencing cycle, each of the 4 individual dNTP solutions (10 μM each; see preparation details above) were flushed sequentially across the chip (15 see @5 ml/min for each nucleotide), with a wash step separating each nucleotide flow. The wash step was performed in two phases: 1) A “through-wash” to flush the nucleotide solution from the fluidic channels and flow cell, and 2) A purge wash, whereby a wash channel by-passes the nucleotide valving apparatus to wash the chip, whilst the next nucleotide solution is simultaneously directed via a purge channel to the waste receptacle. When a nucleotide is incorporated, the subsequent proton release is detected as a change in voltage by the integrated circuit.

### Example 6: Comparison Between Clonal Amplification by RPA and by RPA Followed by LCC

The density of capture oligo on the surface is a factor in determining optimal capture and amplification. Optimal density for RPA was previously found to be obtained using 50 uM capture oligo in coupling reactions. For RPA followed by LCC, the optimum concentration was 2 uM. Data not shown.

Glass slides were prepared, using optimal density of capture oligo (extHDA72R), arranged as spots according to example 1. YdfU and ddl test templates were then hybridised to the capture oligo according to example 2. For all spots, clonal amplification was carried out by an optimised surface phase RPA as per example 3. Fluorescent probes were annealed according to example 4 and the slides imaged. The fluorescent probes were dehybridized with 40 mM NaOH, washed and LCC was performed as described below.

### (a) Hybridization of Molecular Inversion Probes.

A schematic of this approach is given in FIG. 3 and described in detail above. The molecular inversion probe (MIP) was a single stranded polynucleotide having at its 5′ end a 20 nucleotide sequence complementary to the 5′ universal adaptor sequence of the tethered polynucleotide and at its 3′ end a 19 nucleotide sequence which is complementary to the 3′ universal adapter sequence. Between these sequences is a linker sequence of 64 nucleotides. 40 ul of the MIP (6.25e11 copies/ul) in 1× Annealing Buffer (20 mM Tris-HCl pH 7.5, 150 mM sodium chloride, 5 mM magnesium acetate, 0.01% v/v Tween 20 and 5% v/v DMSO) were hybridized through the universal adapter regions of the tethered, clonally amplified oligonucleotides of the 2-D cluster, by incubation at 95° C. for 2 minutes, a step down from 60° C. to 50° C. at a rate of 0.2° C./s before a final incubation at 20° C. for 10 minutes. Following hybridization, the surface was washed twice with 1×RPA wash buffer (0.06% SSC, 0.06% v/v Tween20). The MIP sequence is given below. 5′ and 3′ complementary regions are underlined:

(SEQ ID NO. 7)
/5phos/GACCACAATGAAGCCAAGGCTCGACAGCAGCTTCAACATTCGTTAGTCGAAT
CAGTCCTGTCCGAGGTATTCTTGCGAGTCTAATAAAACGAGACATGCCGAGC

### (b) Extension of the 3′ End of the MIP and Circularization

MIPs were extended downstream of the 3′ terminus to include the sequence of interest from the ddl fragment or ydfU fragment of each surface-conjugated oligo, up to the phosphorylated 5′ of the MIP. The MIP was then ligated to form a circle annealed to the surface-tethered oligo. This process was performed by incubating the surface-tethered oligos with 40 ul of 1× circularization mix (Table 4) at 20° C. for 30 minutes, followed by 60° C. for 10 mins. Following circularization, the surface was washed twice with 1×RPA wash buffer (0.06% SSC, 0.06% v/v Tween 20).

### (c) Rolling Circle Amplification of the Sequence of Interest

Surface-tethered oligonucleotides of the first (2-D) clonal cluster were duplicated by incubation with 40 ul of 1×RCA Mix (Table 5) at 45° C. for 15 minutes, followed by 75° C. for 10 minutes. Following duplication, the surface was washed twice with 60 ul 1×RPA wash buffer (0.06% SSC, 0.06% v/v Tween 20). Following amplification, oligo duplexes were melted by incubation with 40 ul of 40 mM NaOH at 20° C. for 10 minutes and washed twice with 60 ul 1×RPA wash buffer (0.06% SSC, 0.06% v/v Tween 20).

Clonal clusters were visualised by using fluorescent probes (FIG. 5A) and fluorescence was quantified according to example 4 (FIG. 5B). Following rolling circle amplification of the RPA clusters to form a 3-D cluster, there is little difference in fluorescence intensity of 50 uM spots, although there are some areas of increased intensity around the edge of the spots. In contrast, there is significantly increased fluorescence intensity of the 2 uM primer spots. A comparison of spot fluorescence data is given in FIG. 5B. For both YdfU and ddl, RPA+LCC resulted in a 2-3 fold increase in fluorescence intensity.

The example was repeated using an improved MIP as described above, whose sequence is designed to prevent early 3′CR binding and extension. The sequence of the MIP is given below. Simultaneous MIP hybridization and circularization was performed by addition of the MIP to the circularization mix. Heat stable polymerase (Titanium Taq) and ligase (HiFi Ligase) were used to allow for hybridization of the MIP at higher temperatures using the following heating profile—95° C.—2 mins, 55° C.—10 mins, 45° C.-30 mins.

Following circularization, the surface was washed twice with 1×RPA wash buffer (0.06% SSC, 0.06% v/v Tween20). The results displayed in FIG. 8. Fluorescence was again quantified according to example 4 and the distribution of fluorescence across all pixels in an image was plotted. The Y axis is the percentage of pixels having at least the intensity of the X value.

SEQ ID NO: 1
/5Phos/ATGAAGCCAAGGCTGGTGGGTCGACAGCAGCTTCAACATTCGTTAGTCGAATCAG
TCCTGTCCGAGAAAACGAGACATGCC

### Example 7: Sequencing from Clonal Clusters

ISFET chips were coated with ExtHD72R capture oligos according to example 1 for either RPA or RPA+LCC. YdfU template was captured on the chip prepared for RPA according to example 2 and an equimolar mix of YdfU and dll templates were captured on the chip prepared for RPA+LCC. Clonal amplification was carried out according to example 3 for the RCA chip and according to examples 3 and 6 for the RPA+LCC chip. Sequencing from clonal clusters was carried out according to example 5 and results are illustrated in FIG. 6. Graphs C show the signal derived from incorporation of a single nucleotide into the extending sequencing strand during SBS according to example 5. The voltage generated by the ISFET is given in Table 6.

FIG. 6 A provides read length data for RPA (YdfU) and RPA+LCC (YdfU and ddl) clusters. Read data is also given in Table 6.

TABLE 6

| | Relative | ISFET | | Med | Max read | Total length |
|---|---|---|---|---|---|---|
| | Fluorescence | signal | Total | ARL-e | length | of template |
| | Units (RFU) | (mV) | reads | (bp) | (bp) | (bp) |
| RPA | 5-10k | 0.1-0.5 | 1330 | 17 | 39 | 61 |
| (YdfU) | | | | | | |
| RPA + LCC | 10-40k | 1-2 | 66961 | 89 | 101 | 101 |
| (YdfU) | | | | | | |
| RPA + LCC | 10-40k | 1-2 | 13274 | 83 | 97 | 97 |
| (ddl) | | | | | | |

The signal generated from RPA+LCC clusters is superior to that generated from RPA alone and this is reflected in improved read length and read quality.

FIG. 7 illustrates a further comparison between RPA clonal amplification and RPA+LCC clonal amplification. RPA was carried out on capture oligos applied at 25 uM, whilst the RPA+LCC was carried out on oligos applied at 1 uM. The amplifications were carried out on flood filled ISFET chips according to examples 1 to 4 and 6. Capture oligo was extHDA72R and the template was YdfU. The non modified MIP was used (SEQ ID NO:7).

The fluorescence photomicrographs show A—RPA clonally amplified clusters visible as pale cloud-like features (arrowed). The white dots are control spots; B—clonal clusters resulting from RPA followed by LCC amplification. The fluorescence of RPA+LCC clusters (arrowed) is significantly brighter, indicating a higher density of oligonucleotide clones.

The graph, C, illustrates the distribution of fluorescence across all pixels in the image of the chip. The Y axis is the percentage of pixels having at least the intensity of the X value.

### Example 8: Visualisation of Clonality

ExtHD72R capture oligo was spotted at 10 uM onto an ISFET chip as outlined in example 1. Templates (YdfU and ddl equimolar mix) was applied to the capture oligos according to example 2 and subjected to RPA clonal amplification according to example 3. At this point, the YdfU and ddl clusters were visualised using fluorescent probes according to example 4 and imaged. The chip was washed with 40 mM NaOH to remove the fluorescent probe and subjected to LCC according to example 6. The spot was once more labelled with fluorescent probes according to example 4 and re imaged. FIG. 9 shows grey scale images of clonally amplified templates after RPA and RPA+LCC. Arrows indicates individual ddl clonal clusters, the intensity of which is improved in RPA+LCC compared to RPA alone. The right hand images illustrate that these clusters are not detected by the YdfU probe even after LCC.

### Example 9: Generation of YdfU Circles in Solution

The following reagent mixes were used in this example:

TABLE 7
Linear YdfU MIP/Splint Hybridization Mix. This mix contains
all reagents required for the hybridization of the Linear
YdfU MIP and YdfU MIP Splint
YdfU Linear MIP/Splint Hybridisation Mix

| | Stock | | Final | | Volume/ |
|---|---|---|---|---|---|
| Reagent | Concentration | | Concentration | | rxn (ul) |
| Linear YdfU MIP | 100 | uM | 2 | uM | 1.00 |
| YdfU MIP Splint | 100 | uM | 6 | uM | 3.00 |
| Annealing buffer | 5 | X | 1 | X | 10.00 |

| | | | |
|---|---|---|---|
| Water | N/A | N/A | 36.00 |

| | |
|---|---|
| Total Volume | 50.00 |

TABLE 8
Linear YdfU MIP/Splint Ligation Mix. This mix contains
all reagents required for the ligation of the Linear YdfU MIP
into a YdfU circle containing the YdfU template,
flanking regions and MIP linker region.
YdfU Linear MIP/Splint Ligation Mix

| | Stock | | Final | | Volume/ |
|---|---|---|---|---|---|
| Reagent | Concentration | | Concentration | | rxn (ul) |
| T4 DNA ligase buffer | 10 | x | 1 | x | 12.50 |
| T4 DNA ligase | 2000 | U/ul | 200 | U/ul | 12.50 |

| | | | |
|---|---|---|---|
| Hybridisation Mix | N/A | N/A | 50.00 |
| Water | N/A | N/A | 50.00 |

| | |
|---|---|
| Total Volume | 125.00 |

### (a) Linear MIP/Splint Hybridization

50 ul of YdfU Linear MIP/Splint Hybridization Mix was prepared by mixing 6×10¹³ copies of Linear YdfU MIP with 1.8×10¹⁴ copies of YdfU MIP Splint (1:3), in 1× annealing buffer (20 mM Tris-HCl pH 7.5, 150 mM sodium chloride, 5 mM magnesium acetate, 0.01% v/v Tween 20 and 5% v/v DMSO), according to Table 7. The mix was heated to 95° C. for 3-minutes and then incubated at 60° C. for 1 hour.

Linear YdfU MIP
(SEQ ID NO. 8)
/5Phos/ATCCGCTGCGGGTATTACTTAGACCTGTTCTGGTGCCTGAGCTTGGGCTGGTG
GTCCTTAAGCCGGGCCGTGAATCCATACAGATAGACCACAATGAAGCCAAG
GCtcgacagcagcttcaacattcgttagtcgaatcagtcctgtccgaggtattcttgcgagtctaatAAAACGAGACA
TGCCGAGC
Bold sequences are universal binding sites for MIP 5′ and 3′ complementary
regions, lower case sequences are the linker region. Upper case sequences are
YdfU template sequences.
YdfU MIP splint
(SEQ ID NO 9)
AAGTAATACCCGCAGCGGATGCTCGGCATGTCTCGTTTTA

### (b) YdfU Circle Ligation

125 ul of YdfU Linear MIP/Splint Ligation Mix was prepared by adding T4 DNA ligase to the hybridization mix (Table 7) in 1×T4 DNA ligase buffer. The mix was then incubated at 20° C. for 1 hour before a 15-minute heat kill at 75° C.

(c) Extension of the tethered polynucleotide

RPA amplified YdfU clusters on ISFET chips were prepared according to examples 1 to 3. The circular probes were hybridised to the tethered polynucleotides according to example 6(a) and the polynucleotides extended according to example 6(c). Sequencing was then carried out according to example 5. Results are shown in FIG. 10. The signal generated from RPA+LCC clusters using LCC with circles generated in solution rather than on the surface using MIPs, is again superior to that generated from RPA alone and this is reflected in improved read length and read quality.

1. A method for preparing a clonal cluster of a nucleotide sequence of interest comprising:
(i) providing a single stranded polynucleotide comprising the nucleotide sequence of interest, wherein the polynucleotide is tethered to a surface through its 5′ end;
(ii) carrying out a first clonal amplification of the single stranded polynucleotide to provide a first cluster of polynucleotides comprising a plurality of single stranded polynucleotides tethered to the surface through their 5′ ends and comprising the nucleotide sequence of interest; and
(iii) extending tethered single stranded polynucleotides in the first cluster, by the addition of one or more further copies of the nucleotide sequence of interest to provide a cluster of tethered polynucleotides wherein polynucleotides of the cluster comprise a plurality of copies of the nucleotide sequence of interest.

2. A method according to claim 1, wherein the single stranded polynucleotide comprises 5′ and/or 3′ adaptors.

3-5. (canceled)

6. A method according to claim 1, wherein tethered single stranded polynucleotides in the first cluster are extended by rolling circle amplification of the sequence of interest.

7. A method according to claim 1, wherein tethered single stranded polynucleotides in the first cluster are extended by a process comprising:
(a) providing single stranded circular polynucleotide probe comprising a nucleotide sequence which is complementary to the sequence of interest in the first clonal cluster; and
(b) using the circular nucleotide probe as a template, extending a 3′ end of tethered single stranded polynucleotides in the first clonal cluster using a strand displacing polymerase thereby extending the 3′ end of the tethered single stranded polynucleotides by the addition of one or more further copies of the sequence of interest.

8. A method according to claim 7, wherein in the process the single stranded circular polynucleotide probe is synthesized in solution from a linear polynucleotide comprising a complementary copy of the sequence of interest.

9. A method according to claim 7, wherein in the process the single stranded circular polynucleotide probe is synthesized from a Molecular Inversion Probe (MIP) the MIP comprising 5′ and 3′ complementary regions which hybridize to separate nucleotide sequences of the tethered polynucleotide.

10. A method according to claim 7, wherein in the process the single stranded circular nucleotide probe is formed by:
(a) providing a MIP comprising in a 5′ to 3′ direction, a 5′ complementary region, an optional linker region and a 3′ complementary region; wherein the 5′ complementary region and the 3′ complementary are configured to hybridize to separate sequences on the tethered, single stranded polynucleotides;
(b) hybridizing the 5′ complementary region of the MIP and the 3′ complementary region of the MIP to the tethered single stranded polynucleotide;
wherein the 5′ complementary region hybridizes to a sequence that is up stream of the sequence to which the 3′ complementary region hybridizes;
(c) optionally extending the 3′ end of the MIP using the sequence of the single stranded polynucleotide as a template; and
(d) cyclizing the MIP by ligating the optionally extended 3′ end of the MIP to the 5′ end of the MIP;
wherein the sequences of the 5′ complementary region of the MIP and the 3′ complementary region of the MIP are selected such that optional extension and ligation provides a single stranded circular probe comprising a complementary copy of the sequence of interest.

11. A method according to claim 10, wherein in the process the sequence of the 5′ complementary region of the MIP is selected to hybridize only to the 5′ most portion of the SOI or where a 5′ adaptor is present, only to the 5′ adaptor or to the 5′ adaptor and the 5′ most portion of the SOI; and wherein the sequence of the 3′ complementary region is selected to hybridize only to the 3′ most portion of the SOI or where a 3′ adaptor is present, only to the 3′ adaptor or to the 3′ adaptor and the 3′ most portion of the SOI.

12. (canceled)

13. A method according to claim 10, wherein the single stranded polynucleotide comprises 5′ and 3′ adaptors and wherein the 5′ complementary region of the MIP is selected to hybridize only to sequences in the 5′ adaptor and the 3′ complementary region of the MIP is selected to hybridize only to sequences in the 3′ adaptor.

14. A method according to claim 10, wherein in the process the 3′ terminus of the MIP is extended using a polymerase lacking 5′ to 3′ exonuclease activity.

15-18. (canceled)

19. A method according to claim 10, wherein in the process the linking region, where present, comprises one or more functional sequences selected such that, when reproduced in complement the sequence hybridises to a sequencing or other primer or to a capture oligo.

20. A process for preparing a clonal cluster of a single stranded polynucleotide comprising a sequence of interest, the process comprising:
(i) hybridizing a polynucleotide fragment comprising a complementary copy of the sequence of interest to a capture oligo the capture oligo being tethered to a surface through its 5′ end
(ii) extending the 3′ end of the capture oligo using a polymerase to provide a single stranded polynucleotide comprising the sequence of interest, tethered to the surface through its 5′ end;
(iii) clonally amplifying the sequence of interest employing the method of claim 1, to provide a cluster of tethered polynucleotides wherein polynucleotides of the cluster comprise a plurality of copies of the nucleotide sequence of interest.

21. A process for determining a nucleotide sequence of a single stranded nucleotide fragment comprising:
(i) tethering the single stranded fragment to a surface to provide a single stranded polynucleotide comprising a nucleotide sequence of interest tethered to the surface through its 5′ end;
(ii) clonally amplifying the sequence of interest employing the method of claim 1, provide a cluster of tethered polynucleotides wherein polynucleotides of the cluster comprise a plurality of copies of the nucleotide sequence of interest.
(ii) at least partially sequencing SOIs of the cluster to determine a nucleotide sequence of the SOI.

22. A substrate having coupled to its surface a plurality of single stranded polynucleotides arranged as a plurality of clonal clusters, wherein the single stranded polynucleotides within each cluster comprise alternating copies of a first nucleotide sequence and a second nucleotide sequence, wherein the second nucleotide sequence is common to the single stranded nucleotides of each cluster and wherein the first sequence is not.

23. (canceled)

24. A substrate according to claim 22, wherein polynucleotides within each cluster comprises at least 2 copies of the first sequence.

25. A substrate according to claim 22, wherein polynucleotides within each cluster have free 3′ ends.

26-27. (canceled)

28. A substrate according to any of claim 22, which is an ISFET, a glass or silica substrate, an in soluble particulate substrate, or a portion of a microfluidics device adapted to bring the tethered polynucleotides into contact with liquid reagents.

29. A molecular inversion probe for use in the preparation of a clonal cluster of tethered polynucleotides, the molecular inversion probe comprising in a 3′ to 5′ direction:
(a) a 3′ complementary region;
(b) an optional linker region; and
(c) a 5′complementary region
wherein the sequence of the 3′ complementary region is selected to hybridise to a first portion of a tethered polynucleotide and the sequence of the 5′ complementary region is selected to hybridize to a second portion of the tethered polynucleotide separate from the first portion; and wherein the Tm of the 5′ complementary region is higher than the Tm of the 3′ complementary region.

30. (canceled)

31. A method for preparing a clonal cluster of a tethered polynucleotide comprising:
(i) providing a single stranded polynucleotide tethered to a surface through its 5′ end;
(ii) carrying out a first clonal amplification of the single stranded polynucleotide to provide a first cluster of polynucleotides tethered to the surface through their 5′ ends; and
(iii) extending the tethered single stranded polynucleotides in the first cluster, by rolling circle amplification using a MIP according to claim 29.

32. A method according to claim 31 comprising:
(a) contacting the polynucleotides of the first cluster with the MIP;
(b) hybridizing the 5′ complementary region of the MIP to the tethered single stranded polynucleotide at a temperature above the Tm of the 3′ complementary region of the MIP, but at, or below, the Tm of the 5′ complementary region of the MIP, followed by hybridizing the 3′ complementary region of the MIP to the polynucleotide at a temperature at or below the Tm of the 3′ complementary region of the MIP;
(c) optionally extending the 3′ end of the MIP;
(d) cyclizing the molecular inversion probe by ligating the optionally extended 3′ end of the probe to the 5′ end of the probe; and
(e) contacting the cyclized MIP with a strand displacing polymerase to extend the single stranded polynucleotide.
